(19) United States
(12) Patent Application Publication
(10) Pub. No.: US 2015/0231149 A1
(43) Pub. Date: Aug. 20, 2015

(54) SPERM FUNCTION/ACTIVITY

(71) Applicant: University of Dundee, Dundee (GB)

(72) Inventors: Christopher Barratt, Dundee (GB); Paul Wyatt, Dundee (GB)

(21) Appl. No.: 14/350,966

(22) PCT Filed: Oct. 10, 2012
(86) PCT No.: PCT/GB2012/052514

§ 371 Date: Apr. 10, 2014

(30) Foreign Application Priority Data
Oct. 10, 2011 (GB) 1117453.9

(51) Int. Cl. A61K 31/551; A61K 35/52; A61K 31/522; A61K 31/472; A61K 31/519; A61K 31/437

(52) U.S. Cl. CPC A61K 31/551; A61K 31/519; A61K 31/437; A61K 31/522; A61K 31/472; A61K 35/52

(57) ABSTRACT

The present invention concerns phosphodiesterase inhibitors which may be used to modulate sperm motility, activity and/or function. The invention provides modulators of PDE1Ga which may be used to modulate the physiological processes occurring within a sperm cell. The method includes contacting the sperm with a modulator of PDE1Ga activity or function.

### FIELD OF THE INVENTION

The present invention provides compounds, compositions, medicaments and methods for improving sperm motility, function and/or activity. The invention finds particular application in the treatment of men suffering asthenozoospermia and/or conditions caused or contributed to by low sperm motility, function and/or activity. Moreover, the invention may be used to prepare or pre-treat sperm for use in fertility/fertilisation procedures.

### BACKGROUND OF THE INVENTION

Infertility is a significant global problem affecting approximately 15% of couples, amounting to approximately 80 million couples worldwide. Sperm dysfunction (lacking ‘normal’ function) is the single most common cause. Remarkably, there is no drug a man can take, or that can be added to his sperm in vitro to improve fertility. The only option is ART (Assisted Reproductive Technology) which usually consists of a graduation of treatment depending on severity i.e. IUI (Intra Uterine Insemination) for mild, IVF (in vitro fertilisation) for moderate and, ICSI (intra cytoplasmic injection) for men with severe sperm dysfunction. A primary manifestation of sperm dysfunction is poor motility which negatively impacts on successful ART. Sperm motility is critically dependent on cAMP/cGMP. Data from 20 years ago using non selective phosphodiesterase (PDE) inhibitors (PDEIs) showed an increase in motility but this was accompanied by stimulation of acrosome reaction, thus compromising sperm function. The availability of new generation and type specific PDEI's potentially allows us for the first time to specifically modulate sperm motility via targeting of PDEs.

This invention seeks to obviate one or more of the problems associated with the prior art and provides phosphodiesterase inhibitors (PDEI's) that modulate sperm motility, function and/or activity.

### SUMMARY OF THE INVENTION

The present invention concerns phosphodiesterase inhibitors which may be used to modulate sperm motility, activity and/or function.

A first aspect, of this invention provides one or more PDEIs for use in modulating sperm motility, activity and/or function. In one embodiment, the PDEI compounds provided by this invention are provided as compositions for use.

In a second aspect, the invention provides the use of one or more PDEIs in the manufacture of a medicament for modulating sperm motility, activity and/or function.

In a third aspect, the invention provides a method of modulating sperm motility, activity and/or function, said method comprising the step of administering a therapeutically effective amount of one or more PDEIs to the sperm of a subject producing aberrantly motile, active and/or dysfunctional sperm.

Further, embodiments of this invention provide:

(i) one or more PDEIs for use in enhancing, improving or potentiating sperm motility;

(ii) the use of one or more PDEIs for the manufacture of medicaments for enhancing, improving or potentiating sperm motility;

(iii) methods of enhancing, improving or potentiating sperm motility and/or function, said methods comprising the step of administering a therapeutically effective amount of one or more PDEIs to the sperm of a subject producing sperm with reduced motility and/or function.

The PDEI compounds/compositions, medicaments and methods provided by this invention may find further application in the treatment and/or prevention of diseases, conditions and/or syndromes characterised, caused or contributed to by the production of sperm exhibiting aberrant motility, activity and/or function.

In one embodiment, the invention provides compositions, uses, medicaments and methods which may find application in the treatment of asthenozoospermia.

Additionally, the PDEIs provided by this invention may find application in the treatment and/or prevention of conditions and/or syndromes characterised by the production of “dysfunctional” sperm. Sperm “dysfunction” embraces sporadic, acute or chronic instances of any aberrant or abnormal behaviour in sperm.

The various PDEIs, compositions, medicaments and methods described herein may find further application in insemination procedures, fertility programs and/or assisted conception methods—collectively referred to herein as “fertility procedures”. It is to be understood that the term “fertility procedure(s)” encompasses techniques such as intra uterine insemination (described below).

In one embodiment, the invention provides PDEI compounds (and compositions, medicaments and methods comprising/exploiting, the same) for use as pre-treatments to prepare sperm/semen for use in fertility procedures. In such cases, sperm intended for use in a fertility procedure may first be contacted (pre-treated) or incubated with or exposed to, one or more PDEI compound(s), composition(s) or medicament(s) of this invention.

Without wishing to be bound by theory, it is known that conception/fertilisation rates/success may be associated with sperm motility, function and/or activity. Further, reduced fertility/conception success/rates may be associated with reduced sperm motility, function and/or activity. As such, by pre-treating sperm intended to be used in fertility procedures with the PDEI compounds, compositions, medicaments and methods described herein and exploiting the sperm motility, function and/or activity modulatory effects thereof, it may be possible to increase fertilisation/conception rates/success.

In one embodiment, where aberrant (for example reduced) sperm motility, activity and/or function is identified as a factor contributing to fertilisation and/or conception failures, problems or complications, a sperm/semen sample provided by a subject known or predisposed to produce sperm exhibiting low motility and/or aberrant activity and/or function, may be pre-treated with the compound(s), compositions and/or medicaments described herein. In this way it is possible to provide sperm exhibiting improved motility, function and/or activity, suitable for use in fertility procedures.

In view of the above, a further aspect of this invention provides a method of treating/preparing sperm for use in a fertility procedure, said method comprising the steps of:

(a) providing a sperm sample from a subject; and

(b) contacting the sperm sample with one or more PDEI compound(s)

wherein the sperm sample provided by step (b) is suitable or intended for use in a fertility procedure.

Advantageously, the sperm are contacted (or incubated) with one or more PDEI compounds for a period of time and under conditions suitable to effect modulation of sperm motility, function and/or activity.

In one embodiment, prior to use of the sperm sample generated by step (b) above, the compound(s)/composition contacted with the sperm sample, may be removed. For example, the compound(s)/composition contacted with the sperm sample may be removed by subjecting the sample to, for example, one or more washing and/or separation (centrifugation) procedures.

As such, one embodiment of this invention provides a method of preparing sperm for use in a fertility procedure, said method comprising the steps of:

(a) providing a sperm sample from a subject;

(b) contacting the sperm sample with one or more PDEI compound(s); and

(c) removing the one or more PDEI compound(s) from the sperm;

wherein the sperm sample provided by step (c) is suitable or intended for use in a fertility procedure. One of skill will appreciate that removal of the PDEI compound before the sperm is administered to a recipient, prevents unnecessary exposure of the recipient to PDEI compound.

The invention further provides treated sperm for use in fertility procedures, the treated sperm being obtainable by a method comprising:

(a) providing a sperm sample from a subject;

(b) contacting the sperm sample with one or more PDEI compounds; and

(c) optionally removing the one/or more PDEI compounds from the sperm sample to yield a treated sperm for use in fertility procedures.

In one embodiment, the PDEI compounds described herein may be particularly suited to the preparation and/or pre-treatment of sperm/semen samples intended to be used in in intra uterine insemination (IUI) techniques. Briefly an IUI technique may involve a first step of separating highly motile spermatozoa from the seminal plasma. Spermatozoa can be separated using a number of methods including, for example, density gradient separation using colloidal silica suspension. In this method a sperm sample is layered onto a density gradient and centrifuged. The sperm cells associated with high density (ones with a higher degree of motility) are collected at the bottom of the gradient, isolated and washed. The isolated sperm may then be placed in a catheter which is inserted into the vagina and through the cervix. The sperm are then expelled into the uterus. One of skill in this field will appreciate that IUI procedures are normally timed to coincide with ovulation.

PDEI compounds particularly suited for use in the preparation of sperm/semen for insemination (particularly IUI) or as pre-treatments, may exhibit one or more specific characteristics. In one embodiment, the PDEIs provided by this invention may exhibit a prolonged, durable, sustained or residual effect upon sperm motility, function and/or activity—that is to say, the modulatory effects of the various compounds described herein may be observed not only when the compounds are brought into direct contact with sperm, but for a period of time thereafter and/or after the PDEI compound is removed from the sperm. In other words, PDEIs for use in this invention may include those which exert a residual, sustained, lasting, short/long term and/or durable effect upon sperm motility, activity and/or function—in some cases, even after removal of the PDEI from sperm. One of skill will appreciate that PDEI compounds exhibiting this property are particularly useful as pre-treatments for sperm to be used in fertility procedures as not only can the modulatory effects of the compounds be exploited to improve sperm motility, function and/or activity, but the compound can be removed prior to the sperm being used in a fertility procedure, avoiding unnecessary administration of PDEI compounds to the recipient of the sperm.

In relation to sperm, it should be understood that the terms “activity” and/or “function” encompass physiological processes such as, for example, sperm motility, sperm tropism (namely, the tendency of sperm to move towards or away from certain stimuli), and fertilising ability. The terms “activity” and/or “function” may further include processes which occur prior to and during fertilisation and/or interaction with the egg (or membranes/layers thereof)—such processes may include, for example sperm capacitation and acrosomal activity.

With regard to sperm motility, one of skill will appreciate that the term “motility” not only relates to general movement, but may be applied to other aspects of motility such as, for example, the speed of movement of a sperm cell and/or any increase or decrease in the proportion of moving sperm cells in any given population. As such, the PDEIs described herein may be used not only to increase sperm motility, but also to increase the speed of movement of a sperm cell and/or the proportion of moving cells in any given population of sperm.

In view of the above, the invention provides compositions, uses, medicaments and methods which may be used to modulate sperm motility, sperm penetration (for example penetration through vaginal and/or tissue secretions) and/or interactions between sperm and egg.

One of skill will appreciate that the term “modulate” may mean any increase and/or decrease in sperm motility, activity and/or function as compared to (i) the normal or usual motility, activity and/or function of sperm or (ii) the motility, function and/or activity of aberrantly motile, functional and/or active sperm. Normally motile, functioning or active sperm may be produced or provided by individuals known to be free of any disease, condition and/or syndrome which might result in aberrant sperm motility, function and/or activity. Conversely, aberrantly motile, active and/or functional sperm may be provided by a subject having a disease, condition and/or syndrome which affects sperm motility, function and/or activity—such as, for example, asthenozoospermia/oligozoospermia. Sperm fulfilling the criteria listed in this paragraph (and in points (i) and (ii)) may be used as a “control” to assess modulated sperm motility, function and/or activity. Thus any modulation in sperm motility, function and/or activity may be assessed relative to the motility, function and/or activity of a “control” sperm sample.

In one embodiment, the PDEIs provided by this invention are one or more selected from the group consisting of:
(i) substituted pyrimido-[5,4-d]-pyrimidine
(ii) 3,7-dihydro-1-methyl-3-(2-methylpropyl)-1H-purine-2,6-dione (3-isobutyl-1-methylxanthine: IBMX)
(iii) substituted pyrazolo[1,5-a]pyridine derivatives
(iv) Papaverine
(v) 2,3-Benzodiazepine
(vi) analogues, variants and/or derivatives of any of (i)-(v).
   It should be understood that the term “derivative” not only includes the compounds described below but pharmaceutically acceptable salts or esters of any of the compounds encompassed by (i)-(vi) above. It should also be understood that, where appropriate, the invention may relate to compounds in the form of a mixture of enantiomers or of racemic isomers or enriched in an isomer and/or in optically pure form, for example in R or S form.

In one embodiment, the substituted pyrimido-[5,4-d]-pyrimidine for use in this invention is Dipyridamole (otherwise known as persantine: 2,2′,2″,2′″-(4,8-di(piperidin-1-yl)pyrimido[5,4-d]pyrimidine-2,6-diyl)bis(azanetriyl)tetraethanol) or a variant, analogue or derivative thereof. Suitable substituted pyrimido-[5,4-d]-pyrimidines are described in U.S. Pat. No. 3,031,450 together with methods of preparing the same. It is to be understood that all substituted pyrimido-[5,4-d]-pyrimidines described is U.S. Pat. No. 3,031,450 are to be regarded as included within the scope of this invention.

Suitable substituted pyrimido-[5,4-d]-pyrimidines (such as Dipyridamole) may have the following general formula:

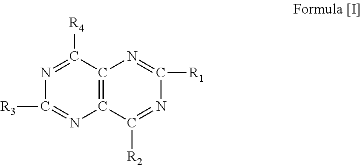

wherein, two three or all four of the substituents R₁ through R₄ are basic groups, that is, primary, secondary or tertiary amino groups; and, if only two or three of said substituents are basic groups, the remaining substituents are hydrogen, halogen, hydroxyl, mercapto, alkyl (for example lower or higher, linear or branched alkyl (including C₁-C₆, C₁-C₅, C₁-C₄, C₁-C₃ or C₁-C₂ or C₇-C₁₀), phenyl, phenoxy, lower alkoxy, lower alkoxy-lower alkoxy, (di-lower alkyl amino)-lower alkoxy, lower alkyl-mercapto, phenyl-mercapto, benzyl-mercapto or carboxy-lower alkyl-mercapto.

As used herein, the term “tertiary amino groups” should also be understood as including substituents wherein the nitrogen moiety is part of the heterocyclic ring, such as, for example, morpholyl, piperidyl, pyrrolidyl, piperazyl, tetrahydropyridyl and tetrahydroquinolyl, which in turn may carry substituents of its own, especially lower alkyl (as described above) substituents.

Dipyridamole may have the formula:

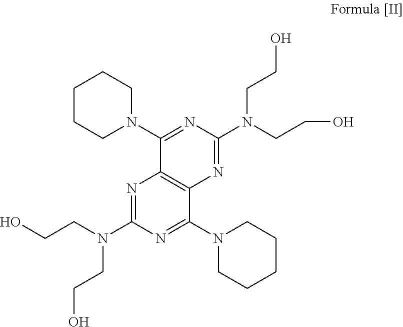 [SMILES: OCCN(CCO)c1nc(N2CCCCC2)c2nc(N(CCO)CCO)nc(N3CCCCC3)c2n1]

In a further embodiment, 3-isobutyl-1-methylxanthine may have the formula:

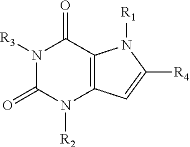

Wherein R₁ is hydrogen, alkyl or methyl

R₂ is hydrogen, alkyl, methyl or isobutyl

R₃ is hydrogen, alkyl or methyl

R₄ is hydrogen, alkyl (lower or higher alkyl—for example linear or branched C₁-C₆ or C₇-C₁₀), phenyl, substituted phenyl, hydroxyl, methyl or (CH₂)ₙ—O—R₅

wherein R₅ is hydrogen, alkyl or methyl.

In one embodiment, the IBMX derivative is 8-MeO-IBMX—otherwise known as MMPX. MMPX may have the formula:

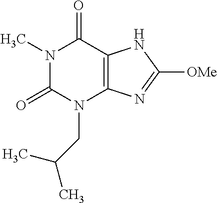 [SMILES: COc1nc2c([nH]1)c(=O)n(C)c(=O)n2CC(C)C]

Substituted pyrazolo[1,5-a]pyridine derivatives for use in this invention are described in U.S. Pat. No. 3,850,941 and suitable compounds may have the formula:

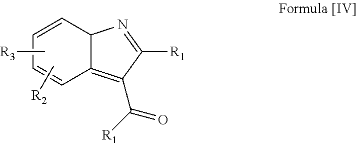

wherein R₁ is hydrogen or a straight or branched alkyl group having from 1 to about 5 carbon atoms (in one embodiment, the branched or straight alkyl group may have 2, 3, 4 or 6 carbon atoms);

R₂ and R₃ are independently hydrogen, methyl, methoxy, halogen, acetoxy or hydroxyl.

In one embodiment, the substituted pyrazolo[1,5-a]pyridine derivative is Ibudilast (2-methyl-1-(2-propan-2-ylpyrazolo[1,5-a]pyridin-3-yl)propan-1-one). Ibudilast may have the formula:

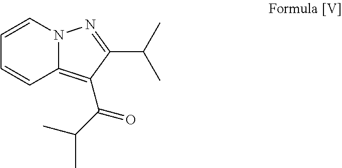 [SMILES: CC(C)C(=O)c1c(C(C)C)nn2ccccc12]

Papaverine may have the following structure:

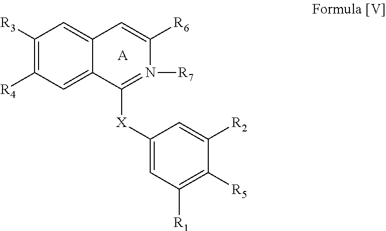

wherein, X is carbonyl or (CH₂)ₙ and n is zero, 1, 2, 3, 4 or 5

R₁, R₂, R₃, R₄, R₅ and R₆ are independently hydrogen, hydroxyl, alkyl (for example a branched or straight alkyl group having 2, 3, 4 or 6 carbon atoms), methyl, methyl or ethoxy; and

R₇ is absent, hydrogen or alkyl (as defined above) or methyl—provided that when R₇ is hydrogen, alkyl or methyl, the double bonds indicated in A are saturated.

Suitable papaverine derivatives may further include pharmaceutically salts thereof.

In one embodiment, papaverine may have the formula:

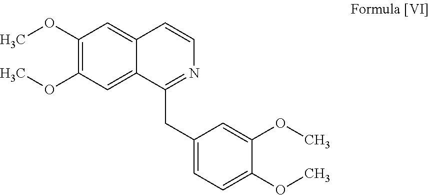

Suitable 2,3-Benzodiazepine are described in US2007/0161628 and compounds for use in this invention may comply with one of the following general formulae (formulae VIIa-VIId):

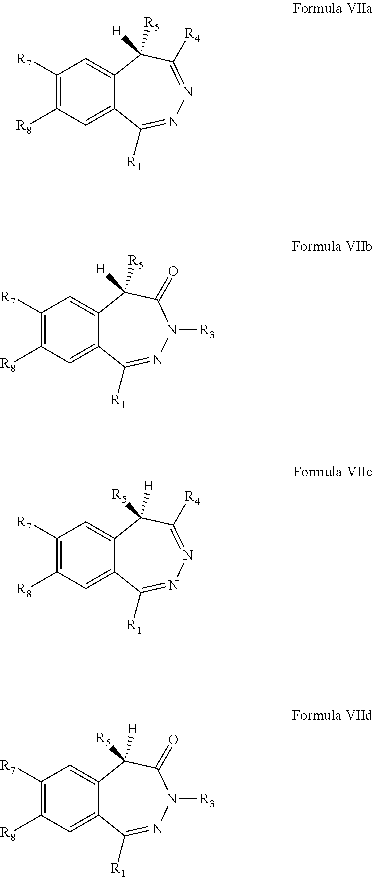

wherein:

R1 and R3, independently of each other, are chosen from a hydrogen atom, a group (C1-C6) alkyl, (C3-C6) cycloalkyl, (C6-C18) aryl, (C6-C18)aryl(C1-C4)alkyl, (C1-C6)alkyl(C6-C18)aryl, (C5-C18) heteroaryl comprising 1 to 3 heteroatoms, or a group OR2, SR2 or NR2R3′ wherein, (i) R2 and R3′, independently of each other, are chosen from a hydrogen atom, and a group (C1-C6) alkyl, (C3-C6) cycloalkyl, (C6-C12) aryl, (C5-C12) heteroaryl comprising 1 to 3 heteroatoms or (ii) R2 and R3′ together form a linear or branched hydrocarbon-based radical containing from 2 to 6 carbon atoms, optionally comprising one or more double bonds and/or optionally interrupted with an oxygen, sulfur or nitrogen atom;

R4 is chosen from a halogen atom, a (C1-C7) alkyl, (C2-C7) alkenyl, (C2-C7) alkynyl, or phenyl group or a group (C—O)R2, OR2, SR2 or NR2R3′ in which R2 and R3′ are as defined above;

R5 is chosen from (C1-C6) alkyl, (C2-C6) alkenyl, (C3-C6) cycloalkyl and (C2-C6) alkynyl groups;

R7 and R8, independently of each other, are chosen from a hydrogen atom, a (C1-C6) alkyl group or a group OR2, SR2 or NR2R3′ in which R2 and R3′ are as defined above;

The alkyl, cycloalkyl, aryl, heteroaryl, alkenyl and alkynyl groups and the hydrocarbon-based chain defined above being optionally substituted with one or more identical or different substituents preferably chosen from a halogen atom, an OH, —O, NO2, NH2, CN, COOH or CF3 group, a (C1-C6) alkoxy group and a group NHCOR2 or CONR2R3′, in which R2 and R3 are as defined above.

In one embodiment, the suitable 2,3-Benzodiazepine compound is Tofisopam (Emandaxin and Grandaxin: 1-(3,4-dimethoxyphenyl)-5-ethyl-7,8-dimethoxy-4-methyl-5H-2,3-benzodiazepine) which may have the following formula:

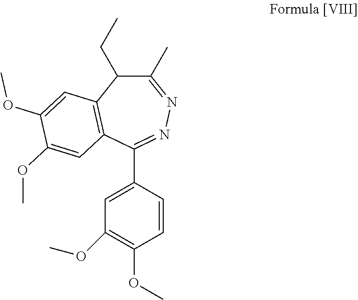 [SMILES: CCC1C(C)=NN=C(c2ccc(OC)c(OC)c2)c2cc(OC)c(OC)cc21]

In view of the above, the present invention provides compositions comprising one or more compounds selected from the group consisting of:
(a) dipyridamole
(b) 8-MeO-IBMX
(c) ibudilast
(d) papaverine
(e) tofisopam
(f) a variant, analogue or derivative of any of (i)-(v)

for use in improving sperm motility or for use in treating and/or preventing asthenozoospermia (or indeed any disease and/or condition which results in the production of aberrantly functional or active sperm).

In one embodiment the various PDEI compounds described herein may be administered (either concurrently or separately) with one or more other compounds or drugs. For example, another of the compounds described herein and/or with another PDEI. In one embodiment, the “other” compound may not be a compound for modulating aberrant sperm function or activity.

The inventors have discovered that each of the PDEI compounds described herein, in particular those listed as (i)-(vi) or (a)-(f) above, exhibit a higher than expected activity. Specifically, the compounds papaverine and tofisopam have been shown to exhibit a greater than expected effect upon sperm motility.

Through experimentation, the inventors have been able to categorise the various PDEI compounds tested as either “mild” or “good” responders—where a “good responder” PDEI compound exerts a greater than expected effect upon sperm motilitly. Some exemplary mild and good responder PDEI compounds are indicated below.

### Structure of Good Responder PDEi (100 μM)

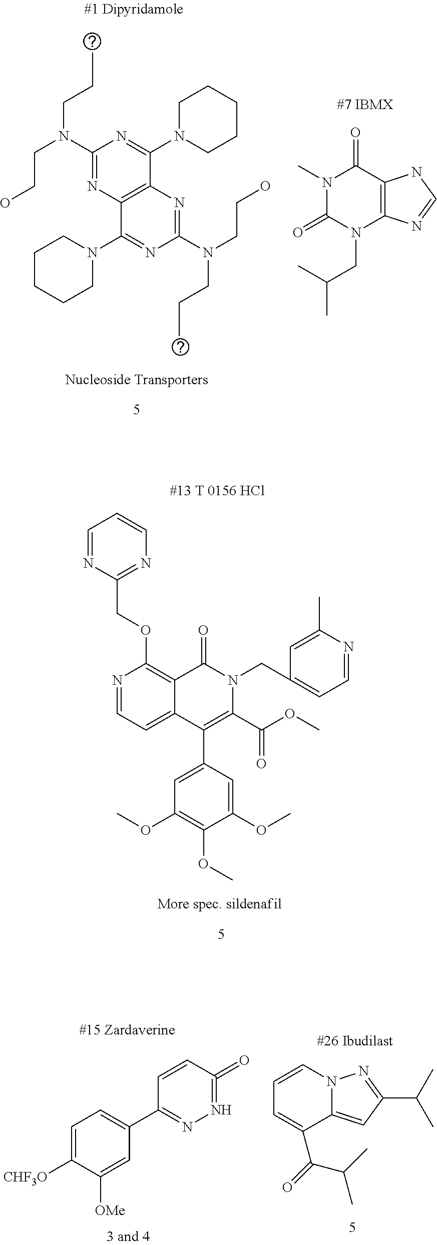

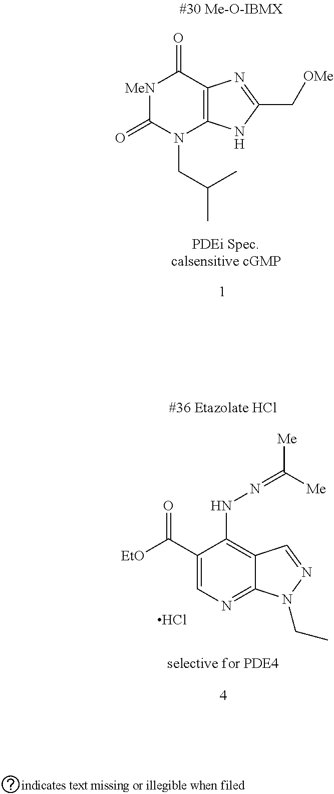 [SMILES: COCc1nc2c(=O)n(C)c(=O)n(CC(C)C)c2[nH]1 ; CCOC(=O)c1cnc2c(cnn2CC)c1NN=C(C)C ; Cl]

### Structure of Mild Responder PDEi (100 μM)

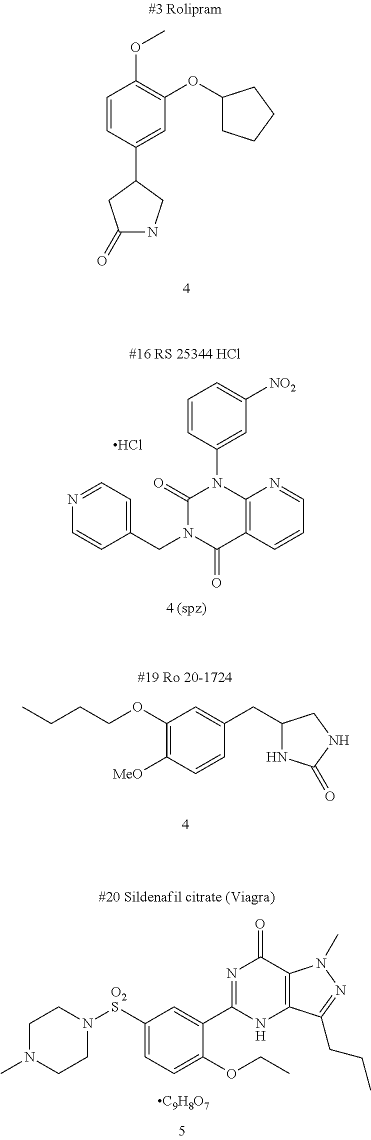

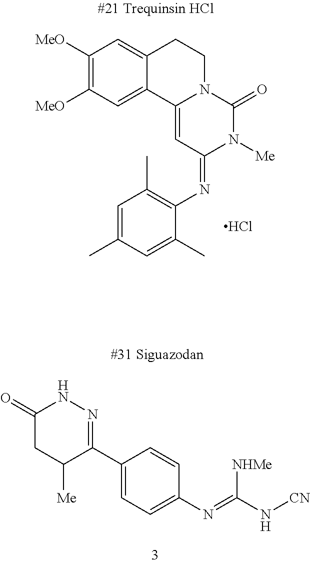 [SMILES: COc1cc2c(cc1OC)-c1c/c(=N\c3c(C)cc(C)cc3C)n(C)c(=O)n1CC2 ; Cl ; CN/C(=N\c1ccc(C2=NNC(=O)CC2C)cc1)NC#N]

### Reproductive and Developmental Biology Group

### Structure of Good Responder PDEi (10 μM)

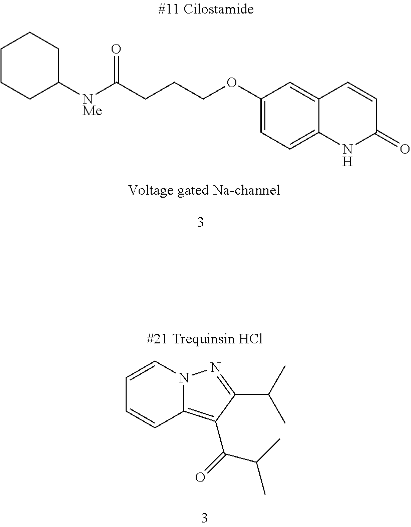 [SMILES: CN(C(=O)CCCOc1ccc2[nH]c(=O)ccc2c1)C1CCCCC1 ; CC(C)C(=O)c1c(C(C)C)nn2ccccc12]

### Structure of Mild Responder PDEi (10 μM)

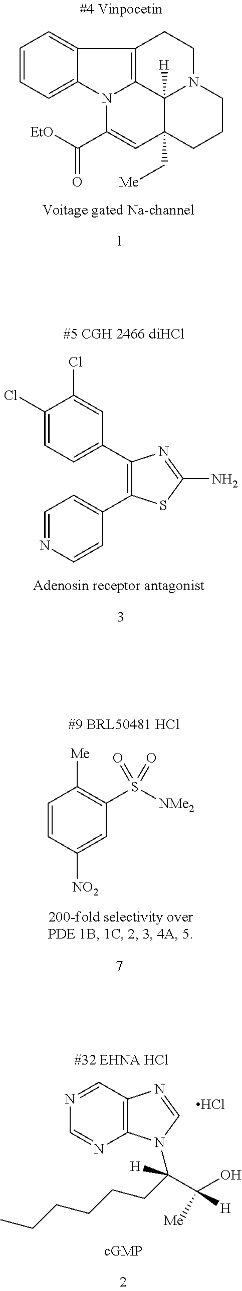

Specifically, the PDEI compounds provided by this invention exert a significant effect upon sperm motility, function, and/or activity and thus represent exemplary selections for the use in modulating sperm activity and/or function. Moreover, when compared to other PDEI compounds, the effect of the PDEI compounds listed herein on sperm motility is significantly greater.

The various PDEI compounds provided by this invention may be formulated as compositions to be administered to sperm as a means of modulating any aberrant motility, function and/or activity and/or to treat or prevent diseases, conditions and/or syndromes characterised by aberrant sperm motility, function and/or activity. Such compositions may comprise, for example, the compounds of the fourth aspect of this invention together with an acceptable diluent, excipient or carrier.

The compositions of this invention may include, one or more additional carrier ingredients such as diluents, freeze-drying adjuvants, buffers, flavouring agents, binders, surface active agents, thickeners, lubricants, preservatives (including anti-oxidants) and the like, and substances included for the purpose of rendering the formulation isotonic with the semen of the intended recipient.

Acceptable carriers are well known to those skilled in the art and include, but are not limited to, 0.1 M and preferably 0.05 M phosphate buffer or 0.8% saline. Freeze-drying adjuvants are well known to those skilled in the art and include mannitol, sucrose, and other complex carbohydrates such as trehalose which raise the glass transition temperature and enhance stability. Additionally, acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In general, a suitable dose of the one or more compounds of the invention may be in the range of about 1 μM to 100 μM with respect to volume of semen of the subject e.g., 1, 5, 10, 25, 50, 100 μM/volume of semen of the subject.

In one embodiment, the suitably formulated dose (the “formulation”) of the one or more compounds provided by this invention is contacted or incubated with a sperm/semen sample intended for use in a fertility procedure—the formulation exerting a motility, function and/or activity modulatory effect on the sperm cells. Prior to use of the sperm in a fertility procedure the one or more compounds and/or compositions brought into contact with the sperm/semen sample may be removed by, for example, washing to provide a treated (and substantially compound/composition free) preparation of sperm ready for use. In one embodiment, the compounds, compositions and/or pharmaceutical compositions provided by this invention are contacted/incubated (in vitro) with a sperm/semen sample provided by a subject and for use in a fertility procedure, for approximately, 5, 10, 20, 30, 40, 50, 60, 90, 120, 150, 180 or 200 mins prior to use. After a suitable period of contact/incubation the compound, composition and/or pharmaceutical composition is removed by, for example, centrifugation and/or washing procedures. Typically, the compounds, compositions and/or pharmaceutical compositions provided by this invention may be contacted or incubated with sperm/semen for use in fertility procedures for a period of time up to about 60 mins, at which point, the compound is removed to provide a treated sperm/semen sample ready for use.

One of skill will appreciate that the identification of highly active compounds presents new possibilities in the field of fertility treatment. For example, while other PDEI compound may have been suggested as able to modulate aspects of sperm activity and/or function, in practice, such compounds could not be used. The compounds provided by this invention present, for the first time, a series of PDEI compounds which represent a viable means for improving sperm motility.

As such, each of compounds (i)-(vi) above represent exemplary compounds to be exploited in the compositions, methods and uses described herein.

In one embodiment, the invention provides dipyridamole or a derivative, analogue or variant thereof, for use in modulating sperm activity and/or function. For example, dipyridamole or a derivative, analogue or variant thereof may be used to modulate, for example enhance or improve sperm motility.

In one embodiment, the invention provides 8-MeO-IBMX or a derivative, analogue or variant thereof, for use in modulating sperm activity and/or function. For example, 8-MeO-IBMX or a derivative, analogue or variant thereof may be used to modulate, for example enhance or improve sperm motility.

In one embodiment, the invention provides ibudilast or a derivative, analogue or variant thereof, for use in modulating sperm activity and/or function. For example, ibudilast or a derivative, analogue or variant thereof may be used to modulate, for example enhance or improve sperm motility.

In one embodiment, the invention provides papaverine or a derivative, analogue or variant thereof, for use in modulating sperm activity and/or function. For example, papaverine or a derivative, analogue or variant thereof may be used to modulate, for example enhance or improve sperm motility.

In one embodiment, the invention provides tofisopam or a derivative, analogue or variant thereof, for use in modulating sperm activity and/or function. For example, tofisopam or a derivative, analogue or variant thereof may be used to modulate, for example enhance or improve sperm motility.

In a further aspect, the present invention provides sperm or semen comprising one or more of the PDEIs listed as (i)-(vi) or (a)-(f) above and/or one or more of the compositions described herein. Mixtures of sperm and PDEI compounds shall be referred to as “sperm/PDEI complexes” hereinafter.

In a further aspect, the present invention provides sperm pre-treated with one or more of the PDEIs listed as (i)-(vi) or (a)-(f) above and/or one or more of the compositions described herein. Additionally or alternatively, the invention provides sperm for use in fertility procedures, said sperm being prepared by a method which comprises the steps of (i) contacting or incubating the sperm with one or more of the PDEIs listed as listed as (i)-(vi) or (a)-(f) above; and (ii) optionally removing the one or more PDEI compounds; to provide a sperm sample ready for use in a fertility procedure (such as, for example IUI).

In one embodiment, the sperm/semen to be contacted/incubated with one or more of the PDEIs described herein may be provided by a subject whose sperm exhibits aberrant function and/or activity—for example low sperm motility. In this way, the addition or supplementation of the sperm sample with one or more PDEIs as described herein, may significantly improve the (i) motility, function and/or activity of the sperm and (ii) the success of a fertility procedure (for example IUI).

Of course it should be understood that while the invention relates primarily to the provision of compounds which improve aspects of sperm motility, activity and/or function, nothing precludes the supplementation (and optional subsequent removal) of healthy (or normal) spermatozoa with one or more of the PDEIs described herein so as to achieve further improved function and/or activity. Sperm exhibiting further improved motility (or higher-motility) may significantly increase the likelihood of successful fertilisation. As such, sperm cells treated with any of the PDEIs provided by this invention may be used in fertility procedures where female subjects experiencing difficulty conceiving are administered (perhaps via IUI techniques), a sperm preparation which has been treated and/or prepared in accordance with aspects and embodiments of this invention. In one embodiment, the sperm for use in a fertility procedure may be “normal” sperm, derived from a healthy donor known to provide sperm which does not exhibit aberrant activity and/or function.

In view of the above, a fifth aspect of this invention provides a method of inseminating a subject, said method comprising the step of:
(i) contacting a sperm sample with one or more of the PDEIs described herein to provide a sperm/PDEI complex;
(ii) optionally removing the one or more PDEIs from the sample to provide a treated sample; and
(iii) inseminating a subject with the treated sample.

Sperm motility and metabolism are dependent upon levels of cyclic AMP (cAMP) and cyclic GMP (cGMP). Phosphodiesterase enzymes degrade cGMP/cAMP and, without wishing to be bound by any particular theory, it is suggested that compounds capable of inhibiting phosphodiesterase enzymes, might be used to promote or enhance sperm function and/or activity including, for example, sperm motility.

There are a number of distinct PDE's family (PDEsI1-11 in mammals) each exhibiting a particular distribution through the various organs, tissues and/or cells of the human or animal body. As such the specific cAMP/cGMP dependent processes occurring within any given organ, tissue or cell may be modulated by increasing and/or decreasing the level of PDE activity in those organs, tissues and/or cells. Further, the degree to which a physiological process is modulated may be affected by the type of PDE targeted.

In this regard, the inventors have discovered that PDE10a is important in the physiological processes occurring within a sperm cell. As such, by targeting PDE10a, it is possible to significantly modulate the physiological processes occurring within a sperm cell.

As such, a seventh aspect of this invention provides modulators of PDE10a activity or function, for use in modulating sperm activity and/or function.

In an eighth aspect this invention provides use of modulators of PDE10a activity or function, for the manufacture of a medicament for modulating sperm activity and/or function.

A ninth aspect, this invention provides a method of modulating sperm activity and/or function, said method comprising the step of administering a therapeutically effective amount of a modulator of PDE10a activity or function, to the sperm of a subject producing aberrantly active and/or dysfunctional sperm.

In addition it should be understood that the invention extends to modulators of PDE10a function or activity for use in preparing sperm for use in fertility procedures as described in more detail above. For example, the PDE10a modulators provided by this invention may be contacted with sperm prior to it being used in a fertility procedure. In other embodiments, the invention provides methods for preparing sperm for use in fertility procedures, wherein the methods comprise contacting sperm/semen samples with PDE10a modulators, for a time period and under conditions suitable to affect modulation of sperm motility, activity and/or function and optionally removing the PDE10a modulator(s) to provide a sperm/semen sample suitable for use in a fertility procedure.

It should be understood that the term “PDE10a activity” or “PDE10a function” embraces any and all of the roles PDE10a plays in an organ, tissue and/or cell. For example, the enzymatic properties of PDE10a are to be regarded as an activity and/or function. As such, the modulators of PDE10a function and/or activity may modulate the enzymatic and/or catalytic activities of the PDE10a enzyme.

As defined above, the term “modulate” should be understood as including any increase and/or decrease in the function and/or activity of PDE10a following contact with a PDE10a modulator as described herein. One of skill will appreciate that the degree of PDE10a modulation may be assessed by comparison with the activity and/or function of a PDE10a not contacted with a PDE10a modulator.

One of skill will appreciated that compounds which inhibit PDE10a function and/or activity may be used to enhance, improve or potentiate sperm functions, activities and/or processes which are PDE10a dependent. In contrast, compounds which increase PDE10a activity or function, may be used to inhibit, suppress or reduce sperm functions, activities and/or processes that are PDE10a dependent.

In one embodiment, the PDE10a modulator is a PDE10a inhibitor. In a further embodiment, the PDE10a modulator is selected from the group consisting of tosifopam; papaverine; and a variant, analogue or derivative thereof. Tosifopam, papaverine and variants, analogues and/or derivatives thereof are described in detail above—those definitions apply here.

It should be understood that any compound which exhibits a specificity and/or affinity for PDE10a and which further modulates the activity of PDE10a, may find application in this invention. Accordingly, the term “modulators of PDE10a” may extend not only to cover classical PDE inhibitors (of the type described herein) but also antibodies which exhibit an affinity, selectivity or specificity for PDE10a and/or oligonucleotides which modulate PDE10a expression.

The term “antibodies” may encompass polyclonal and/or monoclonal antibodies and, for example, IgG, IgM, IgD, IgE and/or IgA isotypes. Furthermore, the term “antibody” should be construed as encompassing fragments such as, for example fragments comprising one or both lights chains and/or one or both heavy chains. Suitable fragments may include those known as Fab fragments, Fab₂ fragments and scfv fragments.

Suitable oligonucleotides may include those known as antisense oligonucleotides complementary to a target sequence. In one embodiment, the antisense oligonucleotides may comprise DNA and/or RNA sequences which are complementary to the DNA or RNA (for example mRNA) sequences encoding PDEs such as, for example PDE10a. In other embodiments, the oligonucleotides for use in this invention may take the form of microRNA (miRNA) or small interfering RNA (siRNA).

In addition to the inhibitors detailed above, the present invention may utilise other forms of inhibitor such as, for example, antisense oligonucleotide or RNAi type inhibitors. Compounds of this type are designed to interfere with the transcription/translation process by targeting specific DNA or RNA sequences. An antisense oligonucleotide may comprise DNA and/or RNA and may be used to significantly reduce or ablate the expression of a particular gene and/or its proteinaceous product. One of skill in the art will appreciate that the invention may encompass the use of oligonucleotides known as microRNA (miRNA) or small/short interfering RNA (siRNA). Such RNA oligonucleotides may be in the form of native RNA duplexes or duplexes which have been modified in some way (for example by chemical modification) to be nuclease resistant. Additionally, or alternatively, the RNA oligonucleotides may take the form of short hairpin RNA (shRNA) expression or plasmid constructs which correspond to or comprise siRNAs of the type described herein. Advantageously, potentially useful RNAi molecules may take the form of double-stranded RNA molecules. In all cases, the antisense DNA or RNA molecules may comprise sequences complementary to the PDE targets described herein—including, for example PDE10a.

It should be noted that for all applications of the antisense technology described herein, techniques and protocols for achieving antisense control of gene expression are known and algorithms such as BIOPREDsi that computationally predict antisense sequences that have an optimal knockdown effect for a given gene may be used to design suitable antisense oligmers.

Compounds for use in modulating sperm motility, function and/or activity may be identified using methods comprising the steps of contacting a test agent with sperm and identifying any modulation in sperm function and/or activity. One of skill will appreciate that the results of such methods may be compared to the results of control experiments in which the function and/or activity of sperm not contacted with a test agent is assessed.

The sperm used in the method described above may comprise aberrantly active/functional (for example low motility) sperm. In which case, the control experiments may be conducted using the same aberrantly functional/active sample, wherein the sample is not contacted with the test agent. In this way, it would be possible to determine whether or not the test agent modulates (for example improves) the motility, function and/or activity of aberrantly functional/active sperm.

Furthermore, the method may include additional steps in which the effect of the test agent on (a) sperm cAMP and cGMP levels, (b) the acrosomal integrity or state (c) DNA integrity and/or (d) sperm penetration is assessed.

In order to determine the effect of a test agent on sperm penetration, the method may involve analysing the progress of sperm through a solution comprising a cervical mucus substitute, including, for example, those comprising methyl cellulose (for example a 0.1-0.5% methyl cellulose solution). By way of example, poorly motile or aberrantly functional or active sperm may not be able to penetrate a solution comprising methylcellulose—as such, if following contact with a test agent, aberrantly functional and/or active sperm is seen to penetrate (or at least further penetrate) a methyl cellulose solution, this may indicate that the test agent modulates sperm penetration.

With regard to the assessment of the acrosome integrity or state, it may be possible to use lectins labelled or conjugated with/to a fluorophore such as, for example, Pisum saivumn lectin (PSA), peanut lectin (PNA) (perhaps labelled with an optically detectable label such as FITC) to distinguish sperm with an intact acrosome from those with no or an incomplete, damaged or breached acrosome. One of skill will appreciate that since lectins such as PSA bind to the outer acrosomal membrane, it would be possible to contact a sperm with a labelled PSA and, by assessing the amount and extent of PSA bound to the acrosome, determine the integrity or state of the acrosome

As such, in addition to assessing the effect of a test agent on sperm motility, it may also be possible to contact sperm with an acrosome binding compound (for example PSA) and, by observing binding between the acrosome binding compound and the acrosome, assess the integrity of the acrosome. In one embodiment, the step of contacting the sperm with an acrosomal binding compound, may occur after the sperm has been contacted with a test agent.

If after contact with a test agent, the acrosome is determined to be intact and not subject of the acrosome reaction, it may be possible to conclude that the test agent has no detrimental effect upon the state of the acrosome or acrosome integrity.

### DETAILED DESCRIPTION

The present invention will now be described in detail with reference to the following Figures which show:

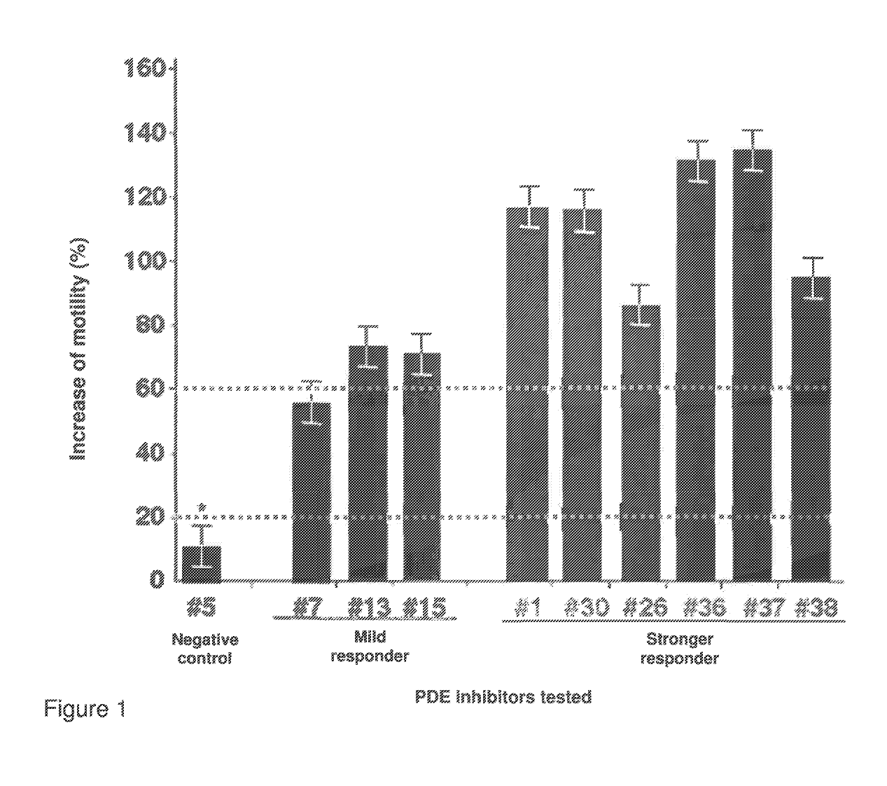

FIG. 1: Sperm motility enhancement (% of increase) following 20 min of PDE inhibitors incubation (100 μM) on human sperm motility (NCM [non capacitated media]; poor fraction; n=3; average±SEM); KEY: #5=CGH 2466, #7=IBMX, #13=T0156 HC, #15=Zardaverine, #1=Dipyridamole, #30=8-MeO-IBMX, #26=Ibudilast, #36=Etazolate HCl, #37=Papaverine, #38=Tofisopam; *: Denotes significant difference to the other, 100% increase=2 fold increase in motility vs 1% DMSO (i.e.: DMSO=15%→treatment 30% motility).

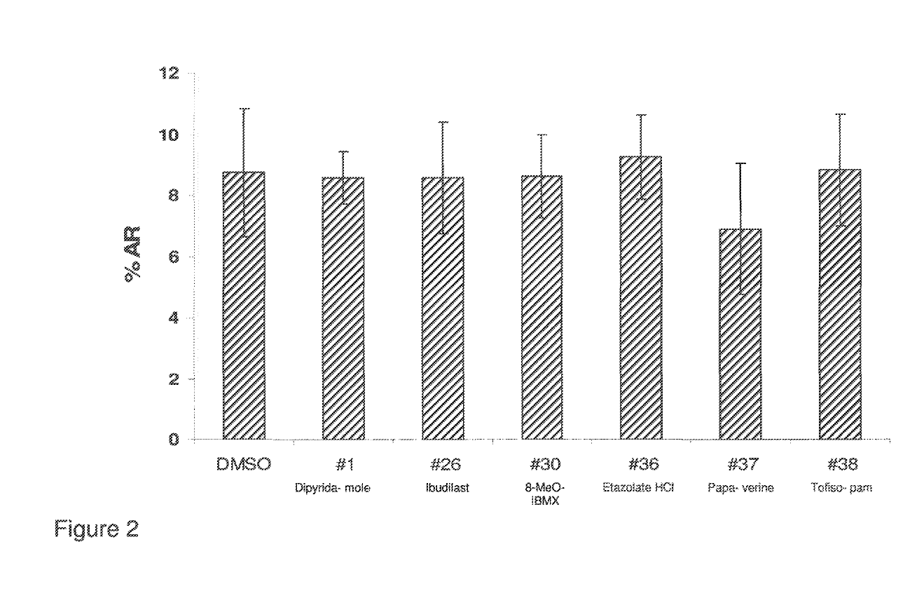

FIG. 2: Effect of PDEi (100 μM) incubation (20 min) on spontaneous acrosome reaction using a NCM (poor motility fraction; n=4; average±SEM). No significant difference was observed among treatment (p=0.6)

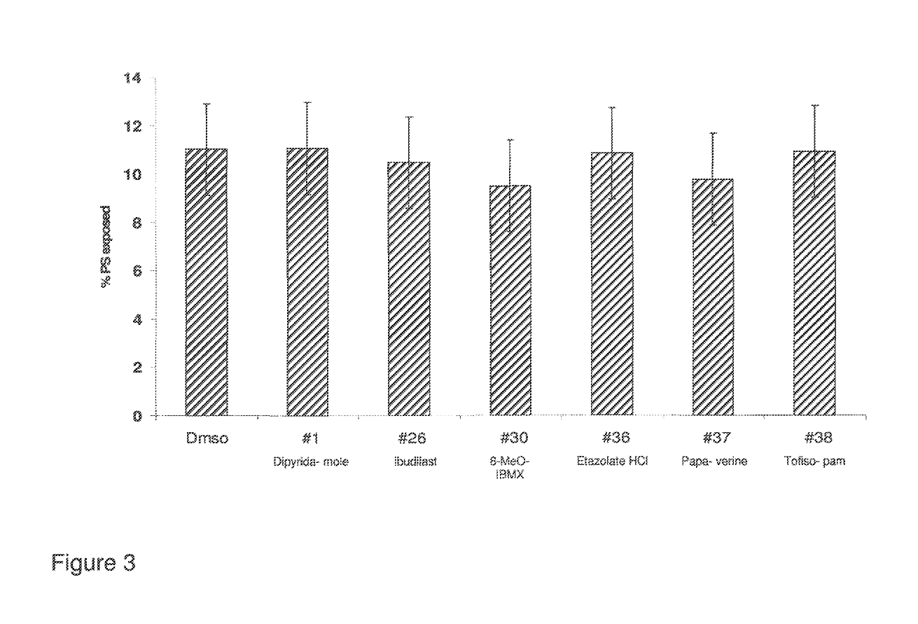

FIG. 3: Effect of PDEi (100 μM) incubation (20 min) on phosphatidylserine exposure detected by annexin V-FITC using a NCM (poor motility fraction; n=4; average±SEM). No significant difference was observed among treatment (p=0.6)

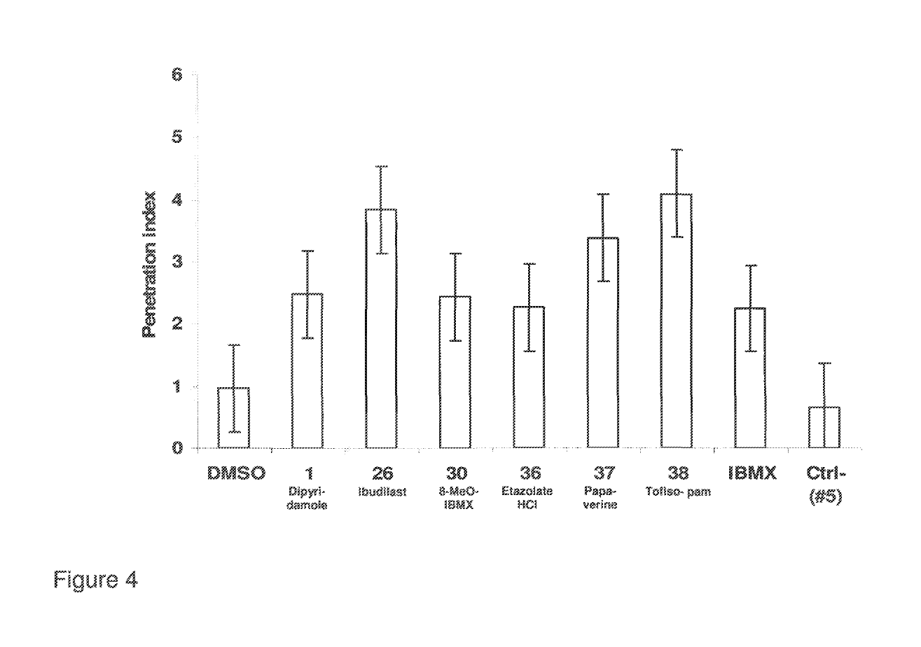

FIG. 4: Sperm penetration assay of poor sperm motility population treated with various PDEi (100 μM) for 20 minutes, sperm cells scored at 1 cm distance (n=6; average±SEM). Note that the best PDEI's to enhance sperm motility in a viscous solution (1% methyl cellulose) were ibudilast, tofisopam and papaverine.

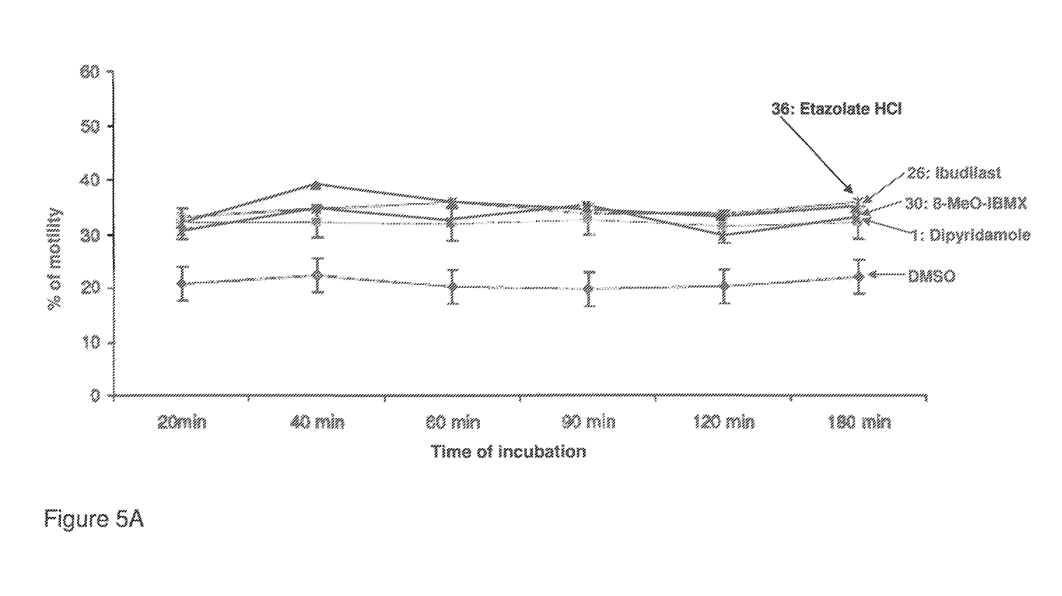

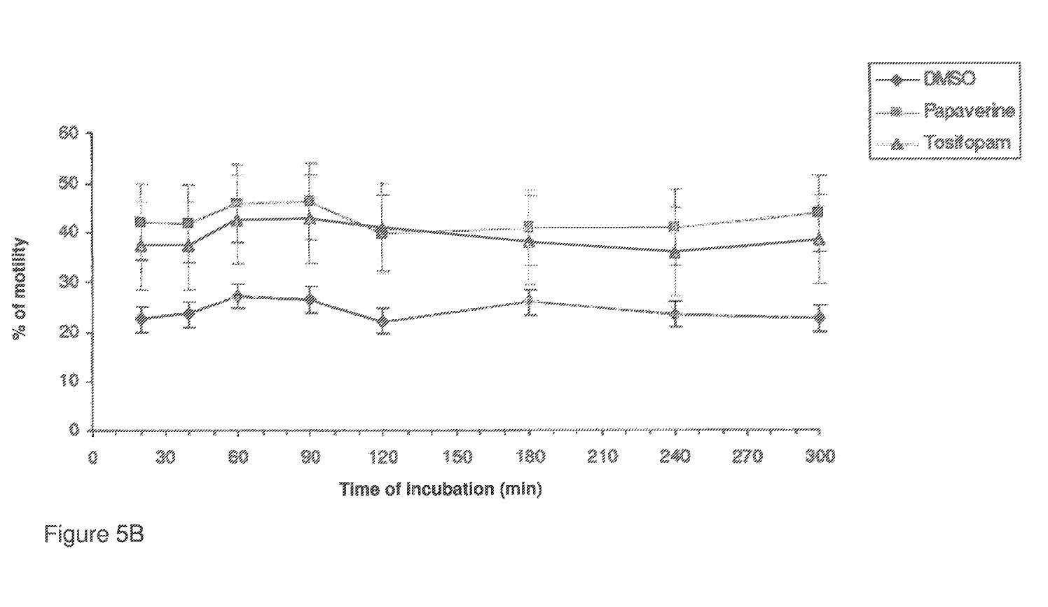

FIG. 5: Longevity of sperm motility enhancement (% total motility) over time using A) dipyridamole, ibudilast 8-methoxy-IBMX, etazolate, B) papaverine and tofisopam. All at 100 μM of PDEI concentration.

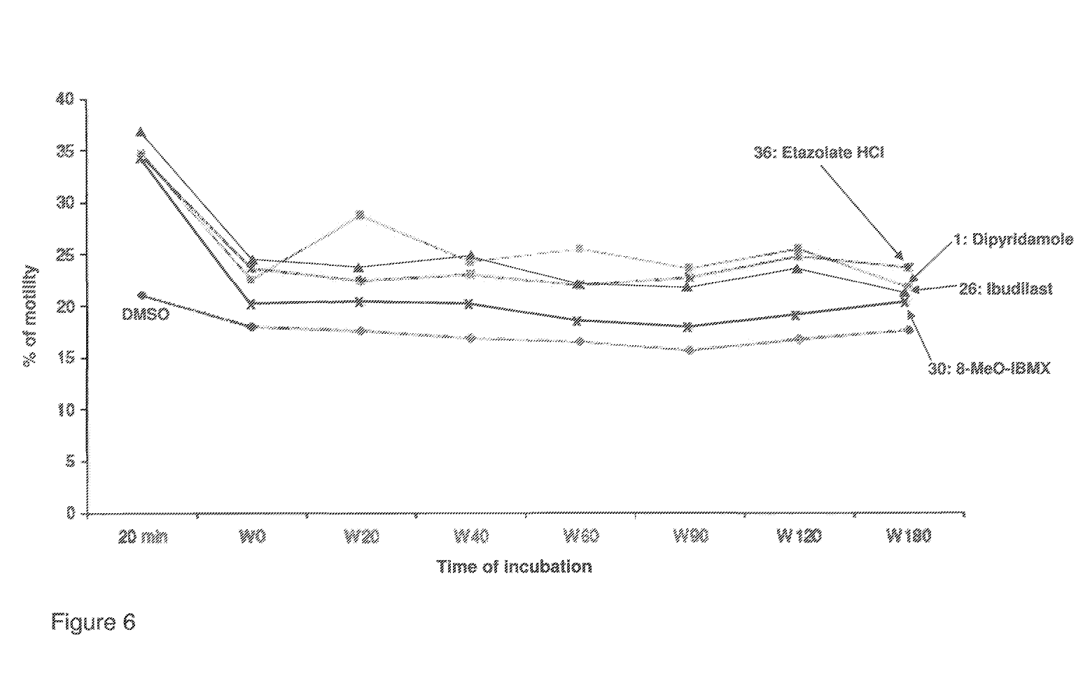

FIG. 6: Percentage of total motility after washing off the PDE inhibitors (100 μM; using poor motility fraction) with four different PDEi after 20 minutes of incubation (n=3)

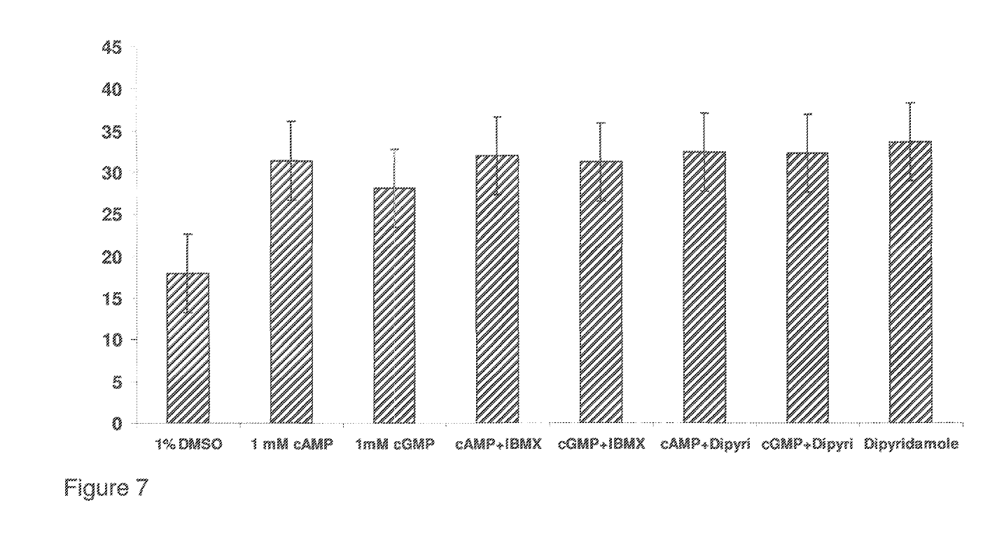

FIG. 7: Effect of cAMP or cGMP (1 mM) on sperm motility enhancement (% Total motility) compared to PDE inhibitors incubation using the poor fraction (NCM; n=5)

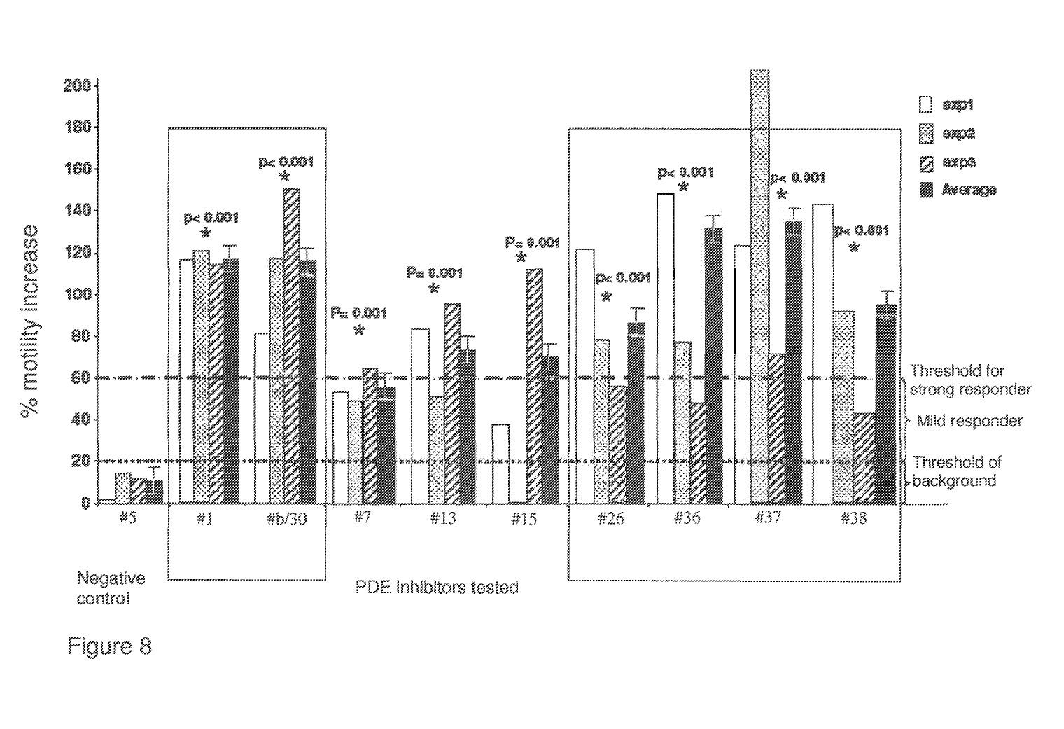

FIG. 8: Sperm motility enhancement (percentage increase) using PDE inhibitors (100 μM final concentration) (poor fraction; average±SD; n=3). 100% increase=2 fold increase in motility vs 1% DMSO (i.e. DMSO=15%→+30%. *: Denotes significant difference to the neg. control. Boxed: most promising PDE inhibitors; Mean±SEM

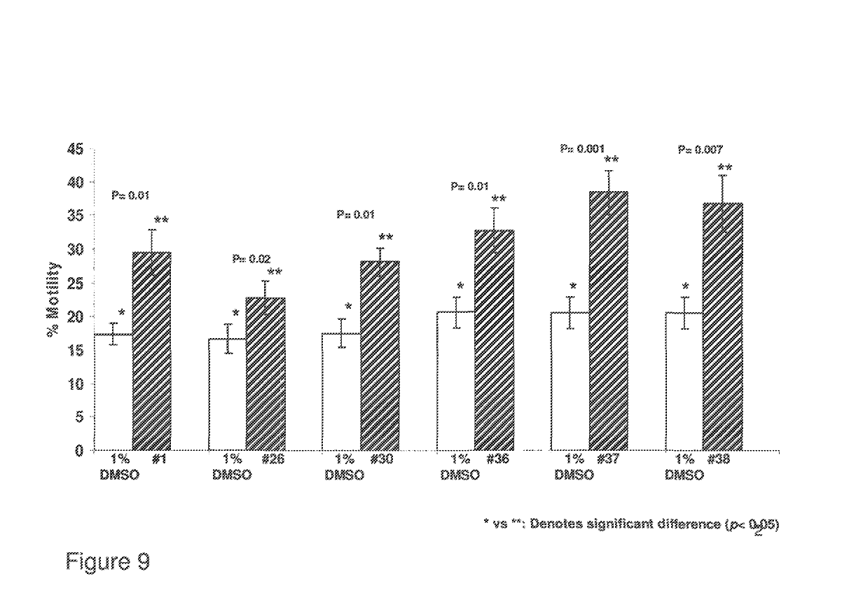

FIG. 9: Sperm motility (%) following incubation with PDE inhibitors (100 μM final concentration) (poor fraction; average±SD; n=6)

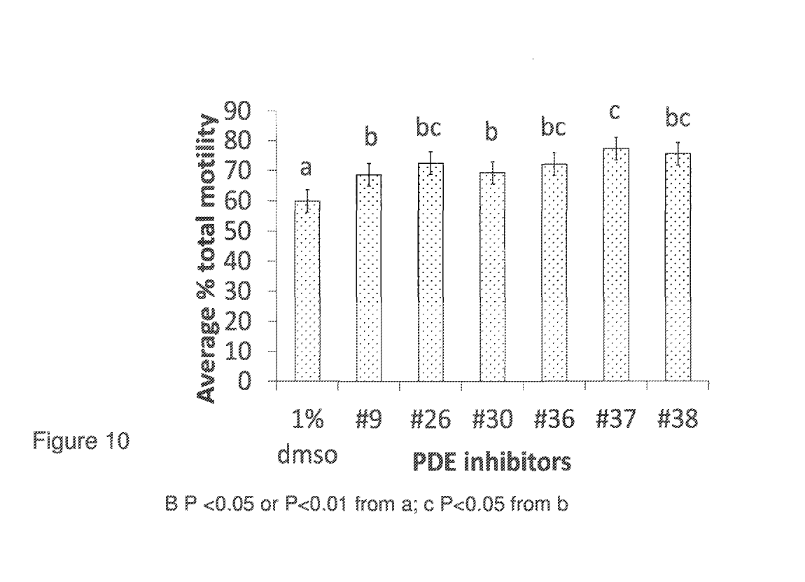

FIG. 10: Sperm motility (%) following incubation with PDE inhibitors (1 μM final concentration) (good fraction; average±SD; n=5). Capacitating conditions.

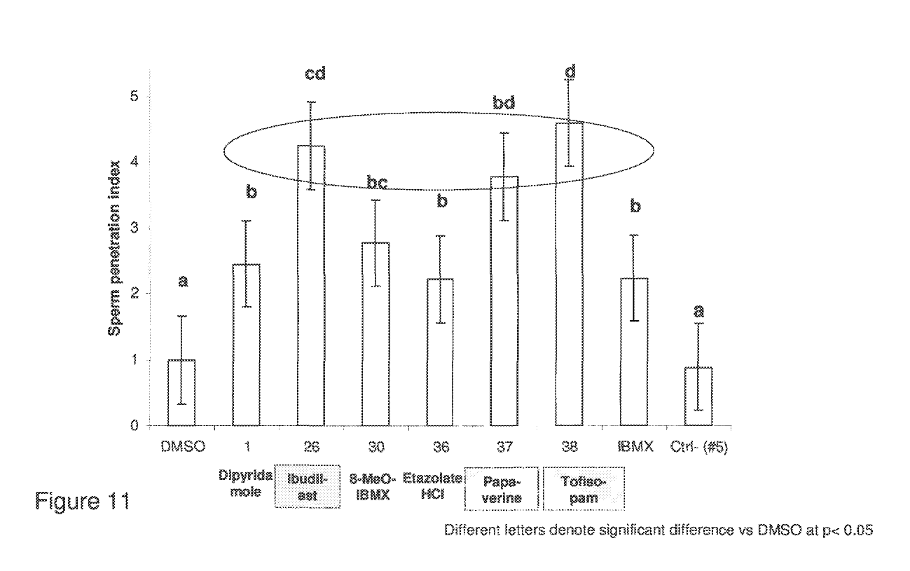

FIG. 11: Sperm penetration assay of poor sperm motility population treated with various PDEi (100 μM) in NCM for 20 min at 1 cm distance (n=8). Different letters denote significant difference vs DMSO at p<0.05

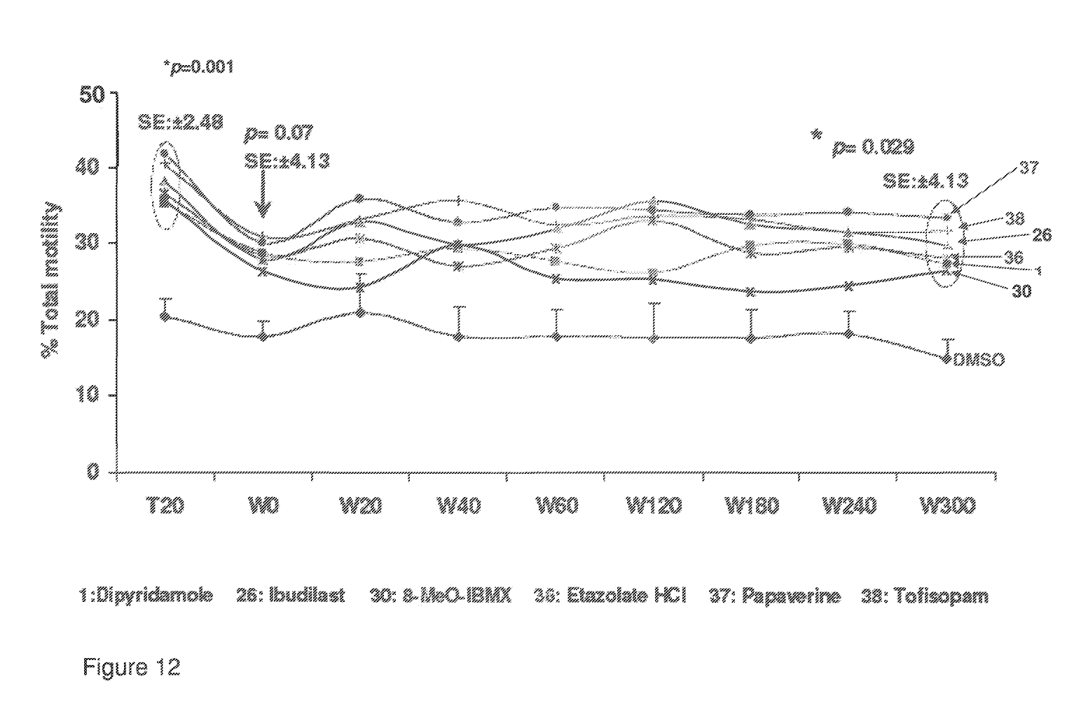

FIG. 12: Total motility after treatment with various PDEi (100 mM, 20 min, NCM) using the poor fraction motility followed by washing out by centrifugation on a period of 5 h (n=4)

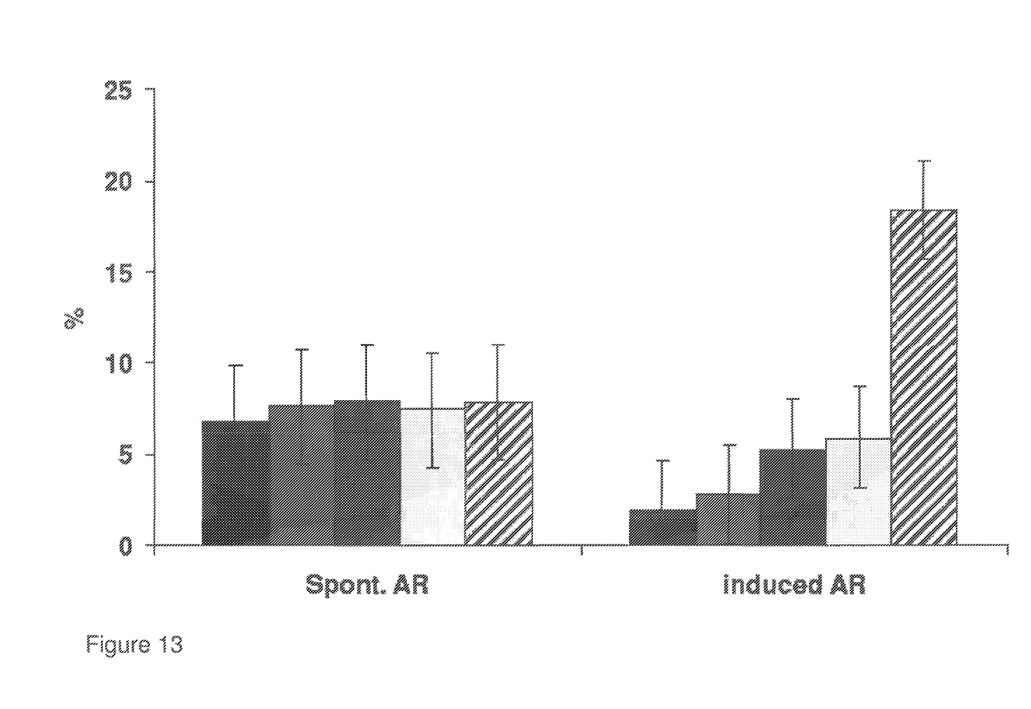

FIG. 13: Effect of sperm incubation in a capacitating medium with 4 different phosphodiesterase inhibitors for 1 h (#26, #37, and #38=0.1 mM; pentoxifylline=3.2 mM) on spontaneous acrosome reaction (AR) or induced acrosome reaction (15 min; 10 mM) using a good sperm population (n=4; average±SEM).

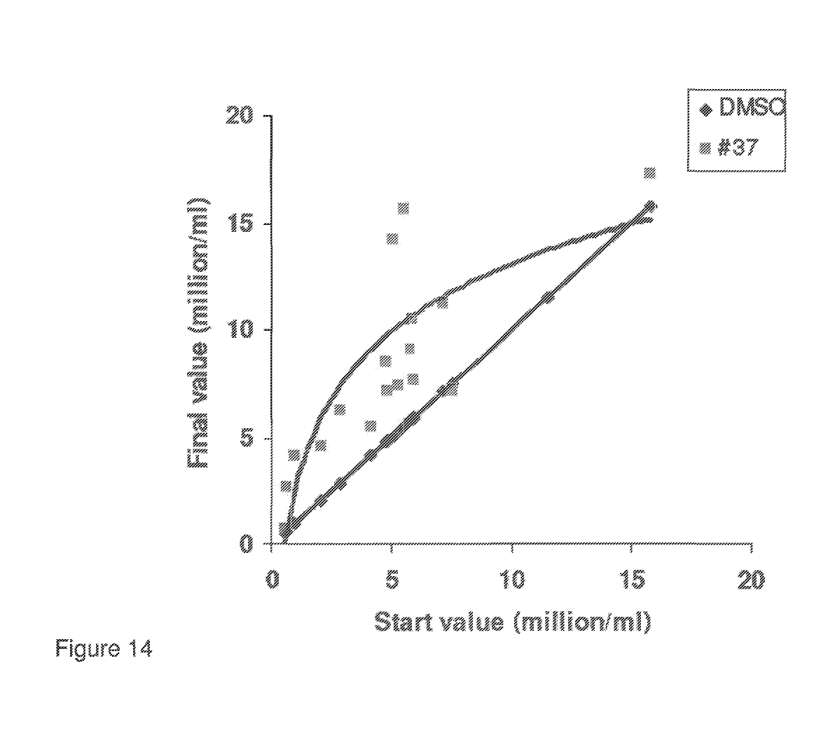

FIG. 14: Effect of Papaverine (#37) on the concentration of motile cells (million/ml) incubated in a NCM medium (20 min) using poor sperm fraction (n=18).

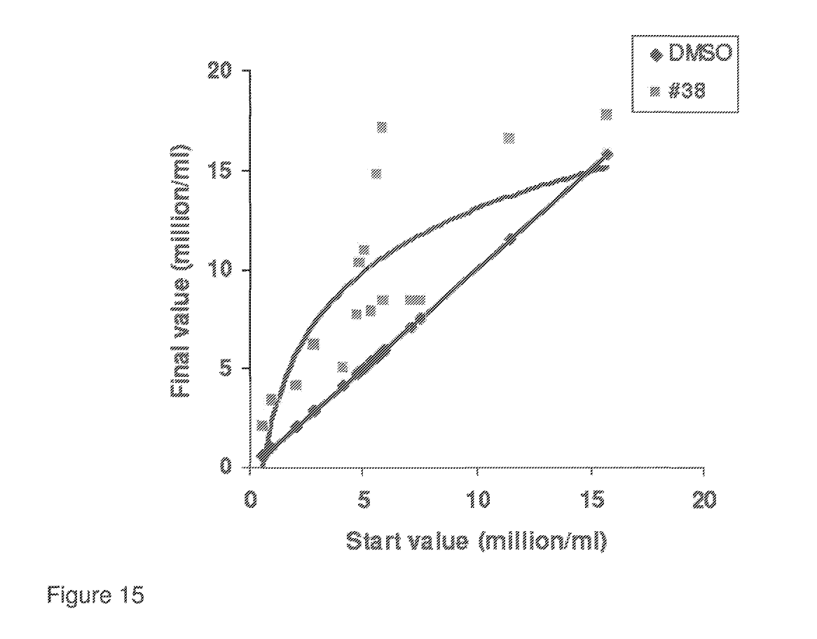

FIG. 15: Effect of Tofisopam (#38) on the concentration of motile cells (million/ml) incubated in a NCM medium (20 min) using poor sperm fraction (n=18)

### MATERIAL AND METHODS

### Chemicals and Experimental Design

Almost all phosphodiesterase inhibitors (PDEi) were purchased from Tocris Bioscience (Bristol, UK) and the other inhibitors from Sigma-Aldrich (Dorset, UK) or Santa Cruz Biotechnology (Heidelberg, Germany) by the Drug Discovery Unit (DDU) at the College Life Sciences (University of Dundee, UK) as shown in Table 1. The DDU coded the PDEi upon arrival and the coded samples were tested for sperm function with a primary focus on sperm motility. All 43 PDEi were analyzed blinded with three independent times for quantitative sperm motility using three different final concentrations (1, 10 and 100 μM). When the experiments were completed the identity of the PDEI was revealed.

### Media and Sperm Preparation

All chemicals were purchased from Sigma-Aldrich. Two different media were used 1-medium not supporting sperm capacitation NCM: 1.8 mM CaCl₂, 5.4 mM KCl, 0.8 mM MgSO₄.7H₂O, 116.3 mM NaCl, 1.0 mM NaH₂PO₄, 5.55 mM D-glucose, 2.73 mM sodium pyruvate, 41.75 mM sodium lactate, 25 mM HEPES and 2-supporting capacitation CM: 1.8 mM CaCl₂, 5.4 mM KCl, 0.8 mM MgSO₄.7H₂O, 116.3 mM NaCl, 1.0 mM NaH₂PO₄, 5.55 mM D-glucose, 2.73 mM sodium pyruvate, 25 mM sodium lactate, 26 mM sodium bicarbonate. Semen samples were obtained from healthy donors (normozoospermic) and samples were produced by masturbation into a sterile plastic container after 2-3 days of sexual abstinence. Two different sperm populations were isolated from the semen using a 40-80% discontinuous density gradient procedure. Briefly, 1 ml of semen was loaded after 30 min of liquefaction at 37° C. on the top of a colloidal silica suspension (Pure Sperm™, Nidacon, Mölndal, Sweden) made of 80% and 40% layered (2 ml each). The density gradient was centrifuged at 300×g for 20 min at room temperature. The motile population was found at the bottom of the 80% fraction and the poor motility population was recovered at the interface of 40-80%, respectively called 80% and 40% fraction. Both fractions were washed using a NCM, cells were isolated by a centrifugation for 5 min at 500×g and the sperm pellet was rediluted using a NCM or CM at ˜20-25×10⁶ cells/ml. Sperm cells from 3-4 different donors were pooled together after sperm preparation in order to obtain enough cells to screen 4-5 PDEi at the same time and additionally to reduced the variability. The first screening was performed with a NCM because it is the conditions normally used in clinic for intrauterine insemination (IUI). Accordingly, NCM is recommended to prevent capacitation before IUI (Björndahl et al., 2010)

### Ethical Approval

Written consent was obtained from each donor and patient in accordance with the Human Fertilisation and Embryology Authority (HFEA) Code of Practice (version 8) under local ethical approval (08/S1402/6) from the Tayside Committee of Medical Research Ethics B.

### Motility Assessment and PDEi Treatment

Following isolation, sperm were mixed with DMSO (vehicle; 1% final concentration) or PDEi [all inhibitors were reconstituted into 1% DMSO]. Sperm cells were incubated for 20 min at 37° C. and the sperm motility was evaluated by using a Computer-Assisted-Sperm-Analysis (CASA, Ceros system [version 12]; Hamilton Thorne, Beverly, Mass.) and 4 chamber slides 20 mM deep (Vitrolife, Sweden). Sperm motility was evaluated from 4 samples per treatment and at least 2,000 sperm cells were analyzed in total (see below). The setting of the CASA used was that established by the manufacturer for human spermatozoa. The percentage of motile cells were identified as follows: rapid cells (percentage of cells exhibiting a VAP>25 μm/s); moderate (VAP of 5-25 μm/s); slow cells were counted as motile cells (VAP<5 μm/s and VSL<11 μm/s) and progressive motility (percentage of cells exhibiting VAP>25 μm/s and straightness>80%).

### Capacitation and Acrosomal Status Evaluation.

Following sperm isolation as described above sperm cells were incubated in absence or in presence of PDEi (100 μM) in NCM for a period of 5 h at 37° C. A sample was taken at the beginning and at every hour until 5 h of incubation. The sample was divided in two, one part was incubated with 10 μM of calcium ionophore A23187 (Sigma) and the other half with the vehicle (1% DMSO), both for 15 min. Sperm capacitation was determined by the ability of the spermatozoa to undergo the acrosome reaction (AR) in response to A23187. Once this incubation was completed, sperm cells were smeared, dried on microscope slides, fixed and permeabilized with 100% methanol incubated at room temperature for 30 min. The percentage of acrosomal reacted-cells was evaluated by using fluroescein-isothiocyanate conjugated pisum sativum lectin as previously described (Morales et at, 1986). Briefly, smeared sperm cells were incubated with PSA-FITC (100 μg/ml) in the dark for 20 min. The slides were washed with Tris-buffered saline (10 mM TRIS-HCl pH 7.4, 150 mM NaCl), mounted with cover slips and at least one to two hundred sperm cells were scored as “acrosome intact” or “acrosome reacted”.

### DNA Integrity and Membrane Selectivity

Phosphatidylserine (PS) is a phospholipid normally located in the inner leaflet of the membrane bilayer. However, PS translocation to the outer leaflet is an event associated with apoptosis related to the loss of DNA integrity (Oosterhuis et al., 2000). In addition, annexin V is known as a phospholipid-binding protein with a high affinity for PS (van Heerde et al., 1995). In these experiments, annexin V conjugated with FITC was used to determine DNA integrity in relation to PDEi incubation. Annexin V does not have the ability to penetrate the membrane and to reach the inner leaflet. Therefore, sperm cells labelled by annexin V performed in a tube (unfixed cells) indicated the presence of PS on the sperm surface demonstrating the loss of the membrane and DNA integrity (Glander and Schaller, 1999).

### Penetration Assay

Methyl cellulose (1% concentration) was used to mimic cervical mucus viscosity and act as a sperm function test (Ivic et al., 2002). Sperm cells were previously treated with PDEi as described above. Glass tubes (5 cm×0.8 cm×2 mm; Vitrocom, NJ, USA) were loaded with methyl cellulose by capillary action (4,000 Sigma M-) prepared in NCM and one extremity was sealed with Cristaseal™ (Hawksley, Sussex, UK). The tubes was incubated with the sperm cells at 37° C. for 1 h 15 min, then the other extremity of the tube was sealed and the number of sperm cells scored at 1 cm and 2 cm using the CASA equipped with a 20× objective magnification. The microscope was adjusted to get the right focus manually at all planes to get the sum of the sperm migrated at this distance. The results are expressed as a penetration index calculated as the number of spermatozoa observed at the site of counting with treatment/the number of spermatozoa without treatment. The speed of sperm population at the migration distance was also analyzed.

### Longevity of Sperm Enhancement in Presence of PDEi

The longevity of the increase of motility produced by PDEi incubation (20 min) was tested over 3-5 hours. The poor sperm population (low motility) was isolated as previously described (FIG. 1) and selected PDEi used to test the longevity over time (dipyridamole, ibudilast, 8-methoxy-IBMX, etazolate, papaverine and tofisopam). In addition, the percentage of total motility has been evaluated after removing the inhibitor by centrifugation (5 min at 300×g).

cAMP and cGMP Effect on Sperm Motility

Sperm cells isolated from the 40% fraction (poor motility) were used to determine the effect of external cyclic nucleotides added during incubation. Sperm solution was mixed with 1 mM of cAMP or 1 mM of cGMP, the sperm motility was recorded by CASA after 20 min of incubation and compared to controls

### Second Messenger Evaluation

Quantification of cAMP and cGMP was evaluated by competitive assay using HTRF technology (CISBIO Bioassay, Codolet, France). In the evaluation of cGMP, the specificity for cAMP was <0.001%. Briefly, sperm cells were incubated with or without PDEi (100 μM) for 20 min as above, then the PDEi was removed by centrifugation (300×g) and spermatozoa were washed twice with NCM. The sperm pellet was gently resuspended in 30 μl (NCM) and 300 μl of 90% ice-cold ethanol was added, and the supernatant was evaporated using a centrifugation-vacuum system. Before evaluation, the cyclic nucleotide was reconstituted in distilled water at 0.5×10⁶ cells/μl equivalent. The evaluation was performed as described by the manufacturer using 5 μl of nucleotide solution.

### Results

TABLE 1
Phosphodiesterase inhibitors screening
for sperm enhancing motility

| | | | Effect on Sperm Motility | | |
|---|---|---|---|---|---|
| exp# | Product Name/Activity | Selectivity | no | Mild | Strong |
| 1 | Dipyridamole | 5/6/8/10/11 | | | X |
| 2 | (R)-(−)-Rolipram | 4; + | X | | |
| 3 | Rolipram | 4 | | X | |
| 4 | Vinpocetine | 1 | | (X) | |
| 5 | CGH 2466 dihydro- | 4 | | (X) | |
| | chloride | | | | |
| 6 | W-7 hydrochloride | Cal-PDE | X | | |
| 7 | IBMX | non-selec | | | X |
| 8 | Milrinone | 3 | X | | |
| 9 | BRL 50481 | 7 | | (X) | |
| 10 | Mesopram | 4 | X | | |
| 11 | Cilostamide | 3 | | | (X) |
| 12 | Pentoxifylline | non-selec | X | | |
| 13 | T 0156 hydro- | 5 | | | X |
| | chloride | | | | |
| 14 | (S)-(+)-Rolipram | 4; − | X | | |
| 15 | Zardaverine | 3 | | | X |
| 16 | RS 25344 hydro- | 4(1, 2, 3) spz | | X | |
| | chloride | | | | |
| 17 | Anagrelide hydro- | 3 | X | | |
| | chloride | | | | |
| 18 | MY-5445 | 5 | X | | |
| 19 | Ro 20-1724 | 4 | | X | |
| 20 | Sildenafil citrate | 5 | | X | |
| 21 | Trequinsin hydro- | 3 ultra | | X | (X) |
| | chloride | cGMP | | | |
| 22 | W-9 hydrochloride | Cal-PDE | X | | |
| 23 | A-7 hydrochloride | Cal-PDE | X | | |
| 24 | Zaprinast | 5/6/9/11 | X | | |
| 25 | Cilostazol | 31 | X | | |
| 26 | Ibudilast | 1a, 3; 2, 4; 5 | | | X |
| 27 | Irsogladine maleate | 4 | X | | |
| 28 | Caffeine | non-selec | X | | |
| 29 | CP 80633 | 4 | X | | |
| 30 | MMPX (8-MeO-IBMX) | 1 | | | X |
| 31 | Siguazodan | 3 | | X | |
| 32 | EHNA hydrochloride | 2 cGMP | | (X) | |
| 33 | Theophylline | non-selec | X | | |
| 34 | YM 976 | 4 | X | | |
| 35 | ICI 63197 | 4 | X | | |
| 36 | Etazolate hydro- | 4 | | | X |
| | chloride | | | | |
| 37 | Pappaverine | 10a | | | X |
| 38 | Tofisopam | 10a | | | X |
| 39 | Nicardipine | Ca chanel | X | | |
| | | bloq | | | |
| 40 | Nimodipine | Ca chanel | X | | |
| | | bloq | | | |
| 41 | Tadalafil | 5 | X | | |
| 42 | Bay-73-6691 | 9a | X | | |
| 43 | Bay-60-7550 | 2a | X | | |

| | | | |
|---|---|---|---|
| Total of 43 PDEI screened | 23 | 10 | 11 |

Summary matrix of patient samples using compounds #1, #26, #30, #36, #37 and #38.
WHO N = WHO normal, WHO borderline = Borderline motility, Low TM and
Low PM = below WHO normal limits for total motility [40%] and progressive motility
[32%]. No entry means not tested, Significance means SD do not overlap.

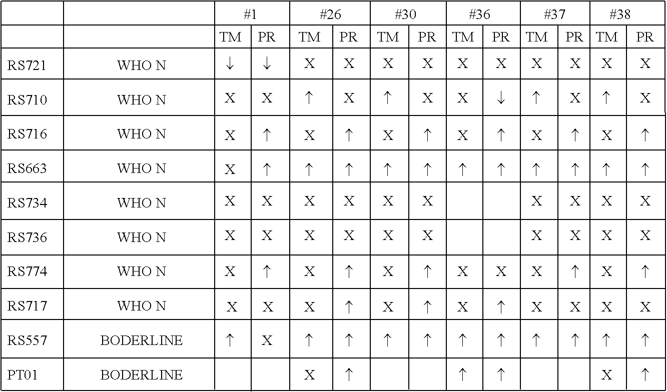

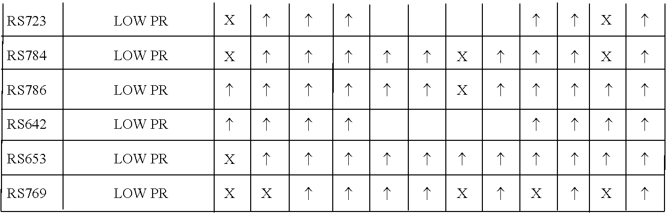

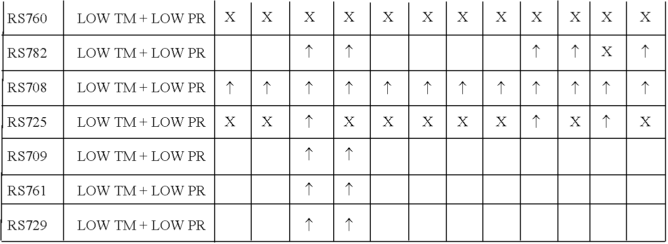

TM = TOTAL MOTILITY WHO 40%
PR = PROGRESSIVE MOTILITY WHO 32%

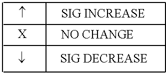

### REFERENCES

Björndahl L, Mortimer D, Barratt C, Castilla J, Menkveld R, Kvist U, Alvarez J, Haugen T. 2010. A practical guide to basic laboratory andrology: Cambridge University Press, Cambridge, UK.
Glander H J, Schaller J. Binding of annexin V to plasma membranes of human spermatozoa: a rapid assay for detection of membrane changes after cryostorage. Mol Hum Reprod 1999; 5:109-115.
Ivic A, Onyeaka H, Girling A, Brewis I A, Ola B, Hammadieh N, Papaioannou S, Barratt C L. Critical evaluation of methylcellulose as an alternative medium in sperm migration tests. Hum Reprod 2002; 17:143-149.
Morales P, Cross N L, Overstreet J W, Hanson F W. Two simple methods for detecting acrosome reaction. Gam Res 1986; 15:213-226.
Oosterhuis G J, Mulder A B, Kalsbeek-Batenburg E, Lambalk C B, Schoemaker J, Vermes I. Measuring apoptosis in human spermatozoa: a biological assay for semen quality? Fertil Steril 2000; 74:245-250.
van Heerde W L, de Groot P G, Reutelingsperger C P. The complexity of the phospholipid binding protein Annexin V. Thromb Haemost 1995; 73:172-179.

1.-23. (canceled)

24. A method modulating sperm motility, activity and/or function or for use in preparing sperm for use in a fertility procedure, said method comprising contacting the sperm with a Modulator of PDE10a activity or function.

25. The method of claim 24, wherein the modulator is selected from the group consisting of;
(i) papaverine;
(ii) tosifopam;
(iii) a variant, analogue or derivative of (i) and/or (ii);
(iv) antibodies which exhibit an affinity, selectivity or specificity for PDE10a; and
(v) oligonucleotides which modulate PDE10a gene expression.

26. The method of claim 25, wherein the one or more modulator compounds exhibit a prolonged, durable, sustained or residual effect upon sperm motility, function and/or activity.

27. A method of modulating sperm motility, activity and/or function, said method comprising contacting sperm with one or more phosphodiesterase inhibitors (PDEIs).

28. The method of claim 27, wherein the one or more PDEIs enhance, improve or potentiate sperm motility, activity and/or function.

29. A method of treating or preventing diseases, conditions and/or syndromes characterised, caused or contributed to by the production of sperm exhibiting aberrant motility, activity and/or function, said method comprising administering an effective amount of one or more PDEIs to a subject in need thereof.

30. The method of claim 29, wherein the disease, condition and/or syndrome is asthenozoospermia.

31. A method of treating/preparing sperm/semen for use in fertility procedures, said method comprising the steps of:
(a) providing a sperm/semen sample from a subject; and
(b) contacting the sperm/semen sample with one or more PDEI compound(s); and
(c) optionally removing the one/or more PDEI compounds from the sperm/semen sample;
wherein the sperm/semen sample provided by steps (b) or (c) is suitable or intended for use in fertility procedures.

32. Treated sperm/semen for use in fertility procedures, the treated sperm/semen being obtainable by a method comprising:
(a) providing a sperm/semen sample from a subject;
(b) contacting the sperm/semen sample with one or more PDEI compounds; and
(c) optionally removing the one/or more PDEI compounds from the sperm/semen sample to yield a treated sperm for use in fertility procedures.

33. The method of claim 31, wherein the fertility procedure comprises an intra uterine insemination (IUI) technique.

34. The treated sperm/semen of claim 32, wherein the fertility procedure comprises an intra uterine insemination (IUI) technique.

35. The method of claim 27, wherein the one or more PDEIs exhibit a prolonged, durable, sustained or residual effect upon sperm motility, function and/or activity.

36. The method of claim 27, wherein the one or more PDEIs are selected from the group consisting of:
(i) Papaverine;
(ii) 2,3-Benzodiazepine;
(iii) substituted pyrazolo[1,5-a]pyridine derivatives;
(iv) substituted pyrimido-[5,4-d]-pyrimidine;
(v) 3,7-dihydro-1-methyl-3-(2-methylpropyl)-1H-purine-2,6-dione (3-isobutyl-1-methylxanthine: IBMX); and
(vi) analogues, variants and/or derivatives of any of (i)-(v).

37. The method of claim 27, wherein the PDEI compound is papaverine having the following structure: 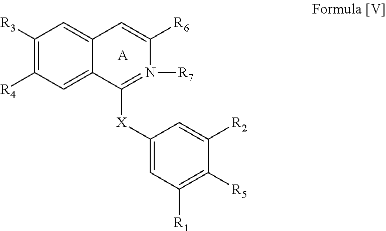
wherein, X is carbonyl or (CH₂)ₙ and n is zero, 1, 2, 3, 4 or 5
R₁, R₂, R₃, R₄, R₅ and R₆ are independently hydrogen, hydroxyl, alkyl (for example a branched or straight alkyl group having 2, 3, 4 or 6 carbon atoms), methyl, methyl or ethoxy; and
R₇ is absent, hydrogen or alkyl (as defined above) or methyl—provided that when R₇ is hydrogen, alkyl or methyl, the double bonds indicated in A are saturated.

38. The method of claim 27, wherein the PDEI compound is 2,3-Benzodiazepine having the following general formulae (formulae VIIa-VIId): 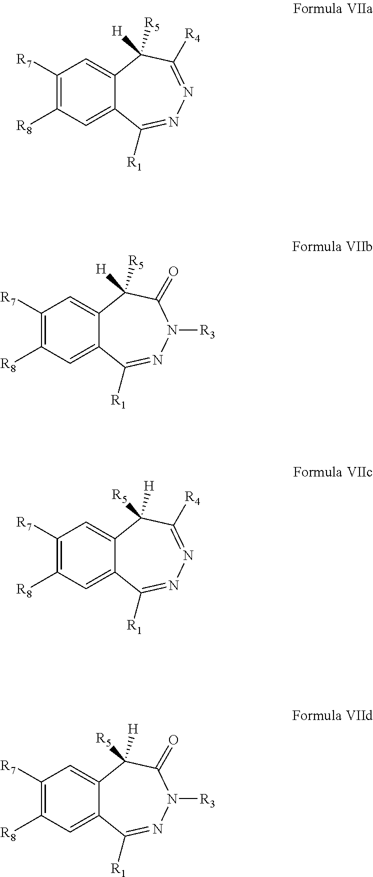
wherein:
R1 and R3, independently of each other, are chosen from a hydrogen atom, a group (C1-C6) alkyl, (C3-C6) cycloalkyl, (C6-C18) aryl, (C6-C18)aryl(C1-C4)alkyl, (C1-C6)alkyl(C6-C18)aryl, (C5-C18) heteroaryl comprising 1 to 3 heteroatoms, or a group OR2, SR2 or NR2R3′ wherein, (i) R2 and R3′, independently of each other, are chosen from a hydrogen atom, and a group (C1-C6) alkyl, (C3-C6) cycloalkyl, (C6-C12) aryl, (C5-C12) heteroaryl comprising 1 to 3 heteroatoms or (ii) R2 and R3′ together form a linear or branched hydrocarbon-based radical containing from 2 to 6 carbon atoms, optionally comprising one or more double bonds and/or optionally interrupted with an oxygen, sulfur or nitrogen atom;
R4 is chosen from a halogen atom, a (C1-C7) alkyl, (C2-C7) alkenyl, (C2-C7) alkynyl, or phenyl group or a group (C—O)R2, OR2, SR2 or NR2R3′ in which R2 and R3′ are as defined above;
R5 is chosen from (C1-C6) alkyl, (C2-C6) alkenyl, (C3-C6) cycloalkyl and (C2-C6) alkynyl groups;
R7 and R8, independently of each other, are chosen from a hydrogen atom, a (C1-C6) alkyl group or a group OR2, SR2 or NR2R3′ in which R2 and R3′ are as defined above;
the alkyl, cycloalkyl, aryl, heteroaryl, alkenyl and alkynyl groups and the hydrocarbon-based chain defined above being optionally substituted with one or more identical or different substituents preferably chosen from a halogen atom, an OH, —O, NO2, NH2, CN, COOH or CF3 group, a (C1-C6) alkoxy group and a group NHCOR2 or CONR2R3′, in which R2 and R3 are as defined above.

39. The method of claim 27, wherein the PDEI compound is a substituted pyrazolo[1,5-a]pyridine derivative having the formula: 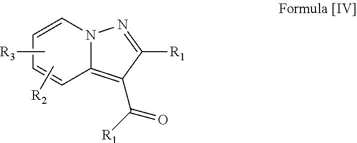
wherein R₁ is hydrogen or a straight or branched alkyl group having from 1 to about 5 carbon atoms (in one embodiment, the branched or straight alkyl group may have 2, 3, 4 or 6 carbon atoms);
R₂ and R₃ are independently hydrogen, methyl, methoxy, halogen, acetoxy or hydroxyl.

40. The method of claim 27, wherein the PDEI compound is a substituted pyrimido-[5,4-d]-pyrimidine having the following general formula: 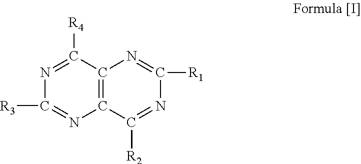
wherein, two three or all four of the substituents R₁ through R₄ are basic groups, that is, primary, secondary or tertiary amino groups; and, if only two or three of said substituents are basic groups, the remaining substituents are hydrogen, halogen, hydroxyl, mercapto, alkyl (for example lower or higher, linear or branched alkyl (including C₁-C₆, C₁-C₅, C₁-C₄, C₁-C₃ or C₁-C₂ or C₇-C₁₀), phenyl, phenoxy, lower alkoxy, lower alkoxy-lower alkoxy, (di-lower alkyl amino)-lower alkoxy, lower alkyl-mercapto, phenyl-mercapto, benzyl-mercapto or carboxy-lower alkyl-mercapto.

41. The method of claim 27, wherein the PDEI compound is 3-isobutyl-1-methylxanthine having the following general formula: 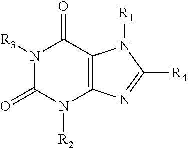
Wherein R₁ is hydrogen, alkyl or methyl
R₂ is hydrogen, alkyl, methyl or isobutyl
R₃ is hydrogen, alkyl or methyl
R₄ is hydrogen, alkyl (lower or higher alkyl—for example linear or branched C₁-C₆ or C₇-C₁₀), phenyl, substituted phenyl, hydroxyl, methyl or (CH₂)ₙ—O—R₅
wherein R₅ is hydrogen, alkyl or methyl.

42. The method of claim 27, wherein the one or more PDEIs are selected from the group consisting of:
(a) papaverine;
(b) tofisopam;
(c) ibudilast;
(d) dipyridamole;
(e) 8-MeO-IBMX; and
(f) a variant, analogue or derivative of any of (i)-(v).

43. Sperm or semen comprising one or more of the PDEIs listed as (i)-(vi) in claim 36.

44. Sperm or semen comprising one or more of the PDEIs listed as (a)-(f) in claim 42.

45. Sperm or semen pre-treated with one or more of the PDEIs listed as (i)-(vi) in claim 36.

46. Sperm or semen pre-treated with one or more of the PDEIs listed as (a)-(f) in claim 42.

47. Sperm or semen for use in a fertility procedure, said sperm or semen being prepared by a method which comprises the steps of (i) contacting or incubating the sperm or semen with one or more of the PDEIs listed as (i)-(vi) in claim 36; and (ii) optionally removing the one or more PDEI compounds; to provide a sperm or semen sample ready for use in a fertility procedure.

48. Sperm or semen for use in a fertility procedure, said sperm or semen being prepared by a method which comprises the steps of (i) contacting or incubating the sperm or semen with one or more of the PDEIs listed as (a)-(f) in claim 42; and (ii) optionally removing the one or more PDEI compounds; to provide a sperm or semen sample ready for use in a fertility procedure.

49. The sperm or semen for use of claim 48, wherein the fertility procedure comprises an intra uterine insemination (IUI) technique.

50. The sperm or semen for use of claim 48, wherein the fertility procedure comprises an intra uterine insemination (IUI) technique.

51. The method of claim 29, wherein the one or more PDEIs are selected from the group consisting of:
(i) Papaverine;
(ii) 2,3-Benzodiazepine;
(iii) substituted pyrazolo[1,5-a]pyridine derivatives;
(iv) substituted pyrimido-[5,4-d]-pyrimidine;
(v) 3,7-dihydro-1-methyl-3-(2-methylpropyl)-1H-purine-2,6-dione (3-isobutyl-1-methylxanthine: IBMX); and
(vi) analogues, variants and/or derivatives of any of (i)-(v).

52. The method of claim 29, wherein the one or more PDEIs are selected from the group consisting of:
(a) papaverine;
(b) tofisopam;
(c) ibudilast;
(d) dipyridamole;
(e) 8-MeO-IBMX; and
(f) a variant, analogue or derivative of any of (i)-(v).

53. The method of claim 31, wherein the one or more PDEIs are selected from the group consisting of:
(i) Papaverine;
(ii) 2,3-Benzodiazepine;
(iii) substituted pyrazolo[1,5-a]pyridine derivatives;
(iv) substituted pyrimido-[5,4-d]-pyrimidine;
(v) 3,7-dihydro-1-methyl-3-(2-methylpropyl)-1H-purine-2,6-dione (3-isobutyl-1-methylxanthine: IBMX); and
(vi) analogues, variants and/or derivatives of any of (i)-(v).

54. The method of claim 31, wherein the one or more PDEIs are selected from the group consisting of:
(a) papaverine;
(b) tofisopam;
(c) ibudilast;
(d) dipyridamole;
(e) 8-MeO-IBMX; and
(f) a variant, analogue or derivative of any of (i)-(v).
